(19) United States
(12) Patent Application Publication
(10) Pub. No.: US 2018/0133212 A1
(43) Pub. Date: May 17, 2018

(54) COMBINATION OF A BCL-2 INHIBITOR AND A BROMODOMAIN INHIBITOR FOR TREATING CANCER

(71) Applicant: Gilead Sciences, Inc., Foster City, CA (US)

(72) Inventors: Jamie Geier Bates, Burlingame, CA (US); Astrid Clarke, Bainbridge Island, WA (US); Thomas F. Kenney, Seattle, WA (US); Saritha Kusam, Foster City, CA (US); Stacey Tannheimer, Seattle, WA (US)

(21) Appl. No.: 15/790,434

(22) Filed: Oct. 23, 2017

Related U.S. Application Data
(60) Provisional application No. 62/416,908, filed on Nov. 3, 2016.

(51) Int. Cl. A61K 31/496; A61K 31/495; A61K 31/5377; A61K 31/444; A61K 31/4709; A61P 35/00

(52) U.S. Cl. CPC A61K 31/496; A61K 31/495; A61P 35/00; A61K 31/444; A61K 31/4709; A61K 31/5377

(57) ABSTRACT

Provided herein are methods and compositions for the treatment of cancer. In particular, the methods include administration of a Bcl-2 inhibitor and a BET inhibitor.

This application claims the benefit of U.S. Provisional Application 62/416,908, filed on Nov. 3, 2016, which is hereby incorporated by reference in its entirety.

### FIELD

The present disclosure relates generally to compositions for treating cancers, and more specifically to the use of Bromodomain and Extra-Terminal motif (BET) inhibitors in combination with B-cell CLL/lymphoma 2 (Bcl-2) inhibitors for treating cancers.

### BACKGROUND

Bcl-2 inhibitors are a class of drugs that function by inhibiting activities of the Bcl-2 protein and are useful in the treatment of cancer. However, some Bcl-2 inhibitors may cause thrombocytopenia and have limited use in clinical treatments (see, e.g., Zhang et al., Cell Death and Differentiation 14:943-951, 2007).

BET inhibitors are a class of drugs with anti-cancer, immunosuppressive, and other effects demonstrated in clinical trials and widely used in research. They reversibly bind the bromodomains of BET proteins BRD2, BRD3, BRD4, and BRDT, and prevent protein-protein interaction between BET proteins and acetylated histones and transcription factors. BET inhibitors include modulators of bromodomain-containing proteins such as the benzimidazole derivatives disclosed in U.S. Pub. No.: 2014/0336190 (Gilead Sciences, Inc.).

### SUMMARY

Provided herein are compositions and methods for treating cancer. In one embodiment, the compositions comprise a Bcl-2 inhibitor and a BET inhibitor. Methods are also provided, in one embodiment, which comprise administering to a human cancer patient:
(i) a therapeutically effective amount of a Bcl-2 inhibitor; and
(ii) a therapeutically effective amount of a BET inhibitor.

In one embodiment, this disclosure provides a composition for use in the treatment of cancer, the composition comprising:
(i) a Bcl-2 inhibitor; and
(ii) a BET inhibitor.

In one embodiment, this disclosure provides a kit comprising:
(i) a pharmaceutical composition comprising a Bcl-2 inhibitor;
(ii) a pharmaceutical composition comprising a BET inhibitor; and
(iii) instructions for use of the Bcl-2 inhibitor and the BET inhibitor in treating cancer.

In one embodiment, the Bcl-2 inhibitor is selected from a group consisting of ABT-199 (venetoclax), ABT-737, ABT-263 (navitoclax), AT-101 (gossypol), apogossypol, TW-37, G3139 (Genasense or oblimersen), obatoclax, sabutoclax, HA14-1, antimycin A, S44563, and combinations thereof. In one embodiment, the Bcl-2 inhibitor is venetoclax.

In one embodiment, the BET inhibitor is a modulator of a bromodomain-containing protein. In one embodiment, the BET inhibitor is an inhibitor of a bromodomain-containing protein. In one embodiment, the BET inhibitor is an inhibitor of bromodomain-containing protein 4 (BRD4).

In one embodiment, the BET inhibitor is a compound of Formula (I):

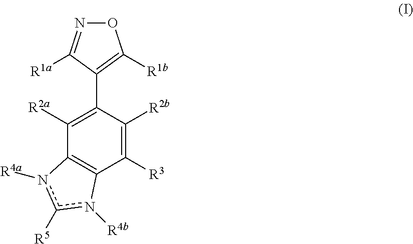

wherein:
one [symbol] is a single bond and the other [symbol] is a double bond;
R^{1a} and R^{1b} are each independently C₁₋₆ alkyl optionally substituted with from 1 to 5 R²⁰ groups;
R^{2a} and R^{2b} are each independently H or halo;
R³ is —B(OH)₂, —B(OR^{a})₂, halo, —C(O)OR^{a}, —NHC(O)OR^{a}, —NHS(O)₂R^{a}, —S(O)₂NR^{a}R^{b}, C₁₋₁₀ alkyl, C₁₋₁₀ alkoxy, amino, C₅₋₁₀ aryl, C₆₋₂₀ arylalkyl, C₁₋₁₀ heteroalkyl, C₅₋₁₀ heteroaryl, or C₆₋₂₀ heteroarylalkyl,
wherein each of C₁₋₁₀alkyl, C₁₋₁₀ alkoxy, amino, C₅₋₁₀ aryl, C₆₋₂₀ arylalkyl, C₁₋₁₀ heteroalkyl, C₅₋₁₀ heteroaryl, or C₆₋₂₀ heteroarylalkyl is optionally substituted with from 1 to 5 R²⁰ groups;
   one of R^{4a} and R^{4b} is selected from the group consisting of H and C₁₋₆ alkyl optionally substituted with from 1 to 5 R²⁰ groups, and the other is absent;
   R⁵ is —C(O)OR^{a}, —NHC(O)OR^{a}, —NHS(O)₂R^{a}, or —S(O)₂NR^{a}R^{b}, H, C₁₋₁₀ alkyl, C₁₋₁₀haloalkyl, C₁₋₁₀ alkoxy, amino, C₅₋₁₀ aryl, C₆₋₂₀ arylalkyl, C₁₋₁₀ heteroalkyl, C₁₋₁₀ heteroaryl, or C₆₋₂₀ heteroarylalkyl,
wherein each of C₁₋₁₀ alkyl, C₁₋₁₀ haloalkyl, C₁₋₁₀ alkoxy, amino, C₅₋₁₀ aryl, C₆₋₂₀ arylalkyl, C₁₋₁₀ heteroalkyl, C₅₋₁₀ heteroaryl, and C₆₋₂₀ heteroarylalkyl is optionally substituted with from 1 to 5 R²⁰ groups;
   each R^{a} and R^{b} is independently selected from the group consisting of H, C₁₋₁₀ alkyl, C₅₋₁₀ aryl, C₆₋₂₀ arylalkyl, C₁₋₁₀ heteroalkyl, C₅₋₁₀ heteroaryl, and C₆₋₂₀ heteroarylalkyl, each of which is optionally substituted with from 1 to 5 R²⁰ groups; and
   each R²⁰ is independently selected from the group consisting of acyl, C₁₋₁₀ alkyl, C₁₋₁₀ alkoxy, amino, amido, amidino, C₅₋₁₀ aryl, C₆₋₂₀ arylalkyl, azido, carbamoyl, carboxyl, carboxyl ester, cyano, guanidino, halo, C₁₋₁₀ haloalkyl, C₁₋₁₀ heteroalkyl, C₅₋₁₀ heteroaryl, C₆₋₂₀ heteroarylalkyl, hydroxy, hydrazino, imino, oxo, nitro, sulfinyl, sulfonic acid, sulfonyl, thiocyanate, thiol, and thione,
   or a pharmaceutically acceptable salt, complex, solvate, prodrug, stereoisomer, mixture of stereoisomers or hydrate thereof.

In one embodiment, the disclosure provides a composition comprising a Bcl-2 inhibitor and Compound A, having the following formula:

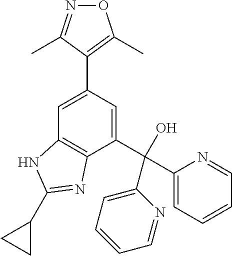 [SMILES: Cc1noc(C)c1-c1cc(C(O)(c2ccccn2)c2ccccn2)c2nc(C3CC3)[nH]c2c1]

or a pharmaceutically acceptable salt, complex, solvate, prodrug, stereoisomer, mixture of stereoisomers or hydrate thereof.

In one embodiment, the disclosure provides a composition comprising venetoclax and Compound A, having the following formula:

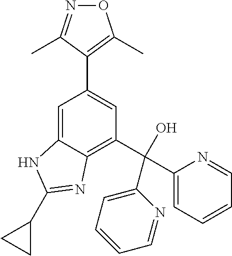 [SMILES: Cc1noc(C)c1-c1cc(C(O)(c2ccccn2)c2ccccn2)c2nc(C3CC3)[nH]c2c1]

or a pharmaceutically acceptable salt, complex, solvate, prodrug, stereoisomer, mixture of stereoisomers or hydrate thereof.

In one embodiment, the disclosure provides a composition comprising venetoclax and a BET inhibitor.

In one embodiment, the compositions and methods disclosed herein are for treatment of a cancer.

In one embodiment, the cancer is a lymphoma. In one embodiment, the cancer is diffuse large B-cell lymphoma (DLBCL). In one embodiment, the cancer is follicular lymphoma (FL).

### BRIEF DESCRIPTION OF FIGURES

FIG. 1 shows induction of apoptosis, inhibition of cell growth, and reduction of Myc protein expression in a number of diffuse large B-cell lymphoma (DLBCL) cell lines in response to treatment with a BET inhibitor (Compound A);

FIG. 2 shows expression levels of apoptotic machinery proteins in DLBCL cell lines in response to Compound A;

FIG. 3 shows growth inhibition in response to venetoclax (ABT-199), a Bcl-2 inhibitor, and apoptosis induction in response to Compound A in the DLBCL cell lines with high and low Bcl-2 levels;

FIG. 4 shows apoptosis induction by Compound A in DLBCL cell lines that are insensitive to venetoclax (ABT-199);

FIG. 5 shows reduction of Myc levels in response to another BET inhibitor (Compound B) in DLBCL cell lines with high expression levels of Bcl-2;

FIG. 6 shows a list of cell lines and synergy scores in response to a combination of venetoclax (ABT-199) and Compound A;

FIG. 7A shows growth inhibition heatmaps of a synergy screen across one FL, one MCL, and seven DLBCL cell lines;

FIG. 7B shows growth inhibition heatmaps of a synergy screen across thirteen DLBCL cell lines;

FIG. 7C shows growth inhibition heatmaps of a synergy screen across eleven MCL cell lines;

FIG. 8 shows improvement in cell growth inhibition by the combination of the inhibitors in DLBCL cell lines; and

FIG. 9 shows growth inhibitory effects of the combination versus single inhibitors at clinically relevant concentrations in DLBCL and MCL cell lines.

### DETAILED DESCRIPTION

The following description sets forth exemplary embodiments of the present technology. It should be recognized, however, that such description is not intended as a limitation on the scope of the present disclosure but is instead provided as a description of exemplary embodiments.

### Definitions

As used in the present specification, the following words, phrases and symbols are generally intended to have the meanings as set forth below, except to the extent that the context in which they are used indicates otherwise.

Recitation of numeric ranges of values throughout the specification is intended to serve as a shorthand notation of referring individually to each separate value falling within the range inclusive of the values defining the range, and each separate value is incorporated in the specification as it were individually recited herein.

Reference to the word “comprise” and variations thereof, such as, “comprises” and “comprising” are to be construed in an open, inclusive sense, that is, as “including, but not limited to.” Further, the singular forms “a,” “an,” and “the” include plural references unless the context clearly dictates otherwise. Thus, reference to “the compound” includes a plurality of such compounds, and reference to “the assay” includes reference to one or more assays and equivalents thereof known to those skilled in the art.

Reference to “about” a value or parameter herein includes (and describes) embodiments that are directed to that value or parameter per se. The term “about X” thus includes description of “X”. In certain embodiments, the term “about” includes the indicated amount ±10%. In other embodiments, the term “about” includes the indicated amount ±5%. In certain other embodiments, the term “about” includes the indicated amount ±1%.

The compound names provided herein are named using ChemBioDraw Ultra 12.0. One skilled in the art understands that the compound may be named or identified using various commonly recognized nomenclature systems and symbols. By way of example, the compound may be named or identified with common names, systematic or non-systematic names. The nomenclature systems and symbols that are commonly recognized in the art of chemistry include, for example, Chemical Abstract Service (CAS), ChemBioDraw Ultra, and International Union of Pure and Applied Chemistry (IUPAC).

A dash at the front or end of a chemical group is a matter of convenience; chemical groups may be depicted with or without one or more dashes without losing their ordinary meaning. A wavy line drawn through a line in a structure indicates a point of attachment of a group. A dashed line indicates an optional bond. Unless chemically or structurally required, no directionality is indicated or implied by the order in which a chemical group is written. For instance, the group “—SO₂CH₂—” is equivalent to “—CH₂SO₂—” and both may be connected in either direction. The prefix “Cᵤ₋ᵥ” indicates that the following group has from u to v carbon atoms, one or more of which, in certain groups (e.g. heteroalkyl, heteroaryl, heteroarylalkyl, etc.), may be replaced with one or more heteroatoms or heteroatomic groups. For example, “C₁₋₆ alkyl” indicates that the alkyl group has from 1 to 6 carbon atoms.

Also, certain commonly used alternative chemical names may or may not be used. For example, a divalent group such as a divalent “alkyl” group, a divalent “aryl” group, etc., may also be referred to as an “alkylene” group or an “alkylenyl” group, an “arylene” group or an “arylenyl” group, respectively.

“Alkyl” refers to any aliphatic hydrocarbon group, i.e. any linear, branched, cyclic, or spiro nonaromatic hydrocarbon group or an isomer or combination thereof. As used herein, the term “alkyl” includes terms used in the art to describe saturated and unsaturated aliphatic hydrocarbon groups with one or more points of attachment, including alkenyl (an aliphatic group containing at least one carbon-carbon double bond), alkylene (a divalent aliphatic group), alkynyl (an aliphatic group containing at least one carbon-carbon triple bond), cycloalkyl (a cyclic aliphatic group), alkylcycloalkyl (a linear or branched aliphatic group attached to a cyclic aliphatic group), and the like. Alkyl groups include, but are not limited to, methyl; ethyl; propyls such as propan-1-yl, propan-2-yl (iso-propyl), and cyclopropyls such as cyclopropan-1-yl, etc.; butyls such as butan-1-yl, butan-2-yl (sec-butyl), 2-methyl-propan-1-yl (iso-butyl), 2-methyl-propan-2-yl (t-butyl), cyclobutan-1-yl; butenes (e.g. (E)-but-2-ene, (Z)-but-2-ene); pentyls; pentenes; hexyls; hexenes; octyls; decyls; cyclopropyl, cyclobutyl, cyclopentyl, cyclohexyl, methylcyclohexyl, spiro[2.4]heptyl, and the like. An alkyl group comprises from 1 to about 10 carbon atoms, e.g., from 1 to 6 carbon atoms. In one embodiment, alkyl is a monovalent, linear or branched, saturated aliphatic hydrocarbon group comprising from 1 to about 10 carbon atoms, e.g., from 1 to 6 carbon atoms.

“Alkenyl” is a subset of“alkyl” and refers to an aliphatic group containing at least one carbon-carbon double bond and having from 2 to about 10 carbon atoms, e.g., from 2 to 6 carbon atoms or 2 to 4 carbon atoms and having at least one site of vinyl unsaturation (>C═C<). Alkenyl groups include ethenyl, propenyl, 1,3-butadienyl, and the like. Alkynyl may have from 2 to about 10 carbon atoms, e.g. from 2 to 6 carbon atoms or 2 to 4 carbon atoms.

“Alkynyl” is a subset of“alkyl” and refers to an aliphatic group containing at least one carbon-carbon triple bond. The term “alkynyl” is also meant to include those groups having one triple bond and one double bond.

“Alkoxy” refers to the group —O-alkyl, wherein the alkyl group may be optionally substituted. Alkoxy includes, by way of example, methoxy, ethoxy, n-propoxy, isopropoxy, n-butoxy, t-butoxy, sec-butoxy, and n-pentoxy.

“Acyl” refers to a group —C(═O)R, where R is hydrogen, alkyl, cycloalkyl, cycloheteroalkyl, aryl, arylalkyl, heteroalkyl, heteroaryl or heteroarylalkyl as defined herein, each of which may be optionally substituted, as defined herein. Representative examples include, but are not limited to formyl, acetyl, cyclohexylcarbonyl, cyclohexylmethyl-carbonyl, benzoyl, benzyloxycarbonyl and the like.

“Amido” refers to both a “C-amido” group which refers to the group —C(═O)NRyRz and an “N-amido” group which refers to the group —NRyC(═O)Rz, wherein Ry and Rz are independently selected from the group consisting of hydrogen, alkyl, aryl, heteralkyl, heteroaryl (each of which may be optionally substituted), and where Ry and Rz are optionally joined together with the nitrogen or carbon bound thereto to form an optionally substituted heterocycloalkyl.

“Amino” refers to the group —NRyRz wherein Ry and Rz are independently selected from the group consisting of hydrogen, alkyl, aryl, heteralkyl, heteroaryl (each of which may be optionally substituted), and where Ry and Rz are optionally joined together with the nitrogen bound thereto to form a heterocycloalkyl or heteroaryl heteroaryl (each of which may be optionally substituted).

“Amidino” refers to the group —C(═NRx)NRyRz where Rx, Ry, and Rz are independently selected from the group consisting of hydrogen, alkyl, aryl, heteralkyl, heteroaryl (each of which may be optionally substituted), and where Ry and Rz are optionally joined together with the nitrogen bound thereto to form a heterocycloalkyl or heteroaryl (each of which may be optionally substituted).

“Aryl” refers to a group with one or more aromatic rings. It may be a single aromatic ring or multiple aromatic rings which are fused together, linked covalently, or linked via one or more such as a methylene or ethylene moiety. Aryl groups include, but are not limited to, those groups derived from acenaphthylene, anthracene, azulene, benzene, biphenyl, chrysene, cyclopentadienyl anion, diphenylmethyl, fluoranthene, fluorene, indane, indene, naphthalene, perylene, phenalene, phenanthrene, pyrene, triphenylene, and the like. An aryl group comprises from 5 to about 20 carbon atoms, e.g., from 5 to 20 carbon atoms, e.g. from 5 to 10 carbon atoms. In one embodiment, aryl is a a single aromatic ring or multiple aromatic rings which are fused together.

“Arylalkyl” (also “aralkyl”) refers to an aryl group attached to an alkyl group. Arylalkyl groups include, but are not limited to, benzyl, tolyl, dimethylphenyl, 2-phenylethan-1-yl, 2-naphthylmethyl, 2-naphthylethan-1-yl, naphthobenzyl, phenylvinyl, diphenylmethyl, and the like. For example, the “arylalkyl” may be attached to the rest of the compound of Formula (I) through the aryl group. Alternatively, the “arylalkyl” may be attached to the rest of the compound of Formula (I) through the alkyl group. Where specific alkyl moieties are intended, the nomenclature arylalkanyl, arylalkenyl and/or arylalkynyl may be used. An arylalkyl group comprises from 6 to about 30 carbon atoms, e.g. the alkyl portion of the arylalkyl group can comprise from 1 to about 10 carbon atoms and the aryl portion of the arylalkyl group can comprise from 5 to about 20 carbon atoms. In some instances an arylalkyl group comprises from 6 to about 20 carbon atoms, e.g. the alkyl portion of the arylalkyl group can comprise from 1 to about 10 carbon atoms and the aryl portion of the arylalkyl group can comprise from 5 to about 10 carbon atoms.

“Azido” refers to the group —N₃.

“Boronic acid” refers to the group —B(OH)₂.

“Boronic acid ester” refers to an ester derivative of a boronic acid compound. Suitable boronic acid ester derivatives include those of the formula —B(OR)₂ where R is hydrogen, alkyl, aryl, arylalkyl, heteroalkyl, or heteroaryl, each of which may be optionally substituted. For example, boronic acid ester may be pinacol ester or catechol ester.

“Carbamoyl” refers to the group —C(O)NR^{y}R^{z} where R^{y} and R^{z} are defined as in “amino” above.

“Carbonyl” refers to the divalent group —C(O)— which is equivalent to —C(═O)—.

“Carboxyl” or “carboxy” refers to —COOH or salts thereof.

“Carboxyl ester” or “carboxy ester” refers to the groups —C(O)OR, wherein R is hydrogen, alkyl, aryl, arylalkyl, heteroalkyl, or heteroaryl, each of which may be optionally substituted. In one embodiment, R is alkyl, aryl, arylalkyl, heteroalkyl, or heteroaryl, each of which may be optionally substituted.

“Cyano” or “carbonitrile” refers to the group —CN.

“Cycloalkyl” is a subset of “alkyl” and refers to a saturated or partially saturated cyclic group of from 3 to about 10 carbon atoms and no ring heteroatoms and having a single ring or multiple rings including fused, bridged, and spiro ring systems. For multiple ring systems having aromatic and non-aromatic rings that have no ring heteroatoms, the term “cycloalkyl” applies when the point of attachment is at a non-aromatic carbon atom (e.g., 5,6,7,8,-tetrahydronaphthalene-5-yl). The term “cycloalkyl” includes cycloalkenyl groups. Examples of cycloalkyl groups include, for instance, adamantyl, cyclopropyl, cyclobutyl, cyclopentyl, cyclooctyl, and cyclohexenyl.

“Guanidino” refers to the group —NHC(═NH)NH₂.

“Halo” or “halogen” refers to fluoro, chloro, bromo and iodo.

“Haloalkyl” refers to substitution of alkyl groups with 1 to 5 or, in one embodiment, 1 to 3 halo groups, e.g., —CH₂Cl, —CH₂F, —CH₂Br, —CFClBr, —CH₂CH₂Cl, —CH₂CH₂F, —CF₃, —CH₂CF₃, —CH₂CCl₃, and the like, and further includes those alkyl groups such as perfluoroalkyl in which all hydrogen atoms are replaced by fluorine atoms.

“Heteroalkyl” refers to an alkyl group in which one or more of the carbon atoms (and any associated hydrogen atoms) are each independently replaced with the same or different heteroatom or heteroatomic group. For example, heteroalkyl may include 1, 2 or 3 heteroatomic groups, e.g. 1 heteroatomic group. Heteroatoms include, but are not limited to, N, P, O, S, etc. Heteroatomic groups include, but are not limited to, —NR—, —O—, —S—, —PH—, —P(O)₂—, —S(O)—, —S(O)₂—, and the like, where R is H, alkyl, aryl, cycloalkyl, heteroalkyl, heteroaryl or cycloheteroalkyl. The term “heteroalkyl” includes heterocycloalkyl (a cyclic heteroalkyl group), alkyl-heterocycloalkyl (a linear or branched aliphatic group attached to a cyclic heteroalkyl group), and the like. Heteroalkyl groups include, but are not limited to, —OCH₃, —CH₂OCH₃, —SCH₃, —CH₂SCH₃, —NRCH₃, —CH₂NRCH₃, and the like, where R is hydrogen, alkyl, aryl, arylalkyl, heteroalkyl, or heteroaryl, each of which may be optionally substituted. A heteroalkyl group comprises from 1 to about 10 carbon and hetero atoms, e.g., from 1 to 6 carbon and hetero atoms.

“Heteroaryl” refers to an aryl group in which one or more of the carbon atoms (and any associated hydrogen atoms) are each independently replaced with the same or different heteroatoms, as defined above. For example, heteroaryl may include 1, 2 or 3 heteroatomic groups, e.g. 1 heteroatomic group. Heteroaryl groups include, but are not limited to, groups derived from acridine, benzoimidazole, benzothiophene, benzofuran, benzoxazole, benzothiazole, carbazole, carboline, cinnoline, furan, imidazole, imidazopyridine, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. A heteroaryl group comprises from 5 to about 20 carbon and hetero atoms in the ring or rings, e.g., from 5 to 20 carbon and hetero atoms, e.g. from 5 to 10 carbon and hetero atoms.

“Heteroarylalkyl” refers to an arylalkyl group in which one or more carbon atoms (and any associated hydrogen atoms) are independently replaced with the same or different heteroatoms, as defined above. For example, heteroarylalkyl may include 1, 2 or 3 heteroatomic groups. Heteroarylalkyl groups include, but are not limited to, groups derived from heteroaryl groups with alkyl substituents (e.g. methylpyridine, dimethylisoxazole, etc.), hydrogenated heteroaryl groups (dihydroquinolines, e.g. 3,4-dihydroquinoline, dihydroisoquinolines, e.g. 1,2-dihydroisoquinoline, dihydroimidazole, tetrahydroimidazole, etc.), isoindoline, isoindolones (e.g. isoindolin-1-one), dihydrophthalazine, quinolinone, spiro[cyclopropane-1,1′-isoindolin]-3′-one, di(pyridin-2-yl)methyl, di(pyridin-3-yl)methyl, di(pyridin-4-yl)methyl, and the like. A heteroarylalkyl group comprises from 6 to about 30 carbon and hetero atoms, for example from 6 to about 20 carbon and hetero atoms.

“Heterocycloalkyl” is a subset of “heteroalkyl” and refers to a saturated or unsaturated cycloalkyl group in which one or more carbon atoms (and any associated hydrogen atoms) are independently replaced with the same or different heteroatom. Heteroatoms include, but are not limited to, N, P, O, S, etc. A heterocycloalkyl group may also contain a charged heteroatom or group, e.g., a quaternized ammonium group such as —N+(R)2- wherein R is alkyl, e.g., methyl, ethyl, etc. Heterocycloalkyl groups include, but are not limited to, groups derived from epoxide, imidazolidine, morpholine, piperazine, piperidine, pyrazolidine, piperidine, pyrrolidine, pyrrolidinone, tetrahydrofuran, tetrahydrothiophene, dihydropyridine, tetrahydropyridine, quinuclidine, N-bromopyrrolidine, N-bromopiperidine, N-chloropyrrolidine, N-chloropiperidine, an N,N-dialkylpyrrolidinium, such as N,N-dimethylpyrrolidinium, a N,N-dialkylpiperidinium such as N,N-dimethylpiperidium, and the like. The heterocycloalkyl group comprises from 3 to about 10 carbon and hetero atoms in the ring or rings. In one embodiment, heterocycloalkyl includes 1, 2 or 3 heteroatomic groups.

“Hydrazino” refers to the group —NHNH₂.

“Hydroxy” or “hydroxyl” refers to the group —OH.

“Imino” refers to the group —C(═NR)— wherein R is hydrogen, alkyl, aryl, arylalkyl, heteroalkyl, or heteroaryl, each of which may be optionally substituted.

“Nitro” refers to the group —NO₂.

“Sulfonyl” refers to the divalent group —S(O)₂—.

“Thiocyanate” refers to the group —SCN.

“Thiol” refers to the group —SH.

“Thione” refers to a thioketone (═S) group.

The terms “optional” or “optionally” mean that the subsequently described event or circumstance may but need not occur, and that the description includes instances where the event or circumstance occurs and instances in which it does not.

“Oxide” refers to products resulting from the oxidation of one or more heteroatoms. Examples include N-oxides, sulfoxides, and sulfones.

“Oxo” refers to a double-bonded oxygen (═O). In compounds where an oxo group is bound to an sp2 nitrogen atom, an N-oxide is indicated.

The term “solvate” refers to an association or complex of one or more solvent molecules and a compound of the disclosure. Examples of solvents that form solvates may include water, isopropanol, ethanol, methanol, dimethylsulfoxide, ethylacetate, acetic acid and ethanolamine.

The term “hydrate” refers to the complex formed by the combining of a compound described herein and water.

The term “prodrug” refers to compounds disclosed herein that include chemical groups which, in vivo, can be converted and/or can be split off from the remainder of the molecule to provide for the active drug, a pharmaceutically acceptable salt thereof, or a biologically active metabolite thereof.

“Stereoisomer” or “stereoisomers” refer to compounds that differ in the chirality of one or more stereocenters. Stereoisomers include enantiomers and diastereomers. The compounds may exist in stereoisomeric form if they possess one or more asymmetric centers or a double bond with asymmetric substitution and, therefore, can be produced as individual stereoisomers or as mixtures. Unless otherwise indicated, the description is intended to include individual stereoisomers as well as mixtures. The methods for the determination of stereochemistry and the separation of stereoisomers are well-known in the art (see, e.g., Chapter 4 of Advanced Organic Chemistry, 4th ed., J. March, John Wiley and Sons, New York, 1992).

“Tautomer” refers to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Substituted” (as in, e.g., “substituted alkyl”) refers to a group wherein one or more hydrogens have been independently replaced with one or more substituents including, but not limited to, alkyl, alkenyl, alkynyl, alkoxy, acyl, amino, amido, amidino, aryl, azido, carbamoyl, carboxyl, carboxyl ester, cyano, guanidino, halo, haloalkyl, heteroalkyl, heteroaryl, heterocycloalkyl, hydroxy, hydrazino, hydroxyl, imino, oxo, nitro, sulfinyl, sulfonic acid, sulfonyl, thiocyanate, thiol, thione, or combinations thereof. Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to -substituted aryl-(substituted aryl)-substituted aryl. For example, in one embodiment, when a group described above as being “optionally substituted” is substituted, that substituent is itself unsubstituted. Similarly, it is understood that the above definitions are not intended to include impermissible substitution patterns (e.g., methyl substituted with 5 fluoro groups or heteroaryl groups having two adjacent oxygen ring atoms). Such impermissible substitution patterns are well known to the skilled artisan. When used to modify a chemical group, the term “substituted” may describe other chemical groups defined herein. For example, the term “substituted aryl” includes, but is not limited to, “arylalkyl.” Generally, substituted groups will have 1 to 5 substituents, 1 to 3 substituents, 1 or 2 substituents or 1 substituent. Alternatively, the optionally substituted groups of the invention may be unsubstituted.

It is understood that combinations of chemical groups may be used and will be recognized by persons of ordinary skill in the art. For instance, the group “hydroxyalkyl” would refer to a hydroxyl group attached to an alkyl group. A great number of such combinations may be readily envisaged.

Compounds of a given formula described herein encompass the compound disclosed and all pharmaceutically acceptable salts, esters, stereoisomers, tautomers, prodrugs, hydrate, solvates, and deuterated forms thereof, unless otherwise specified.

“Pharmaceutically acceptable” refers to compounds, salts, compositions, dosage forms and other materials which are useful in preparing a pharmaceutical composition that is suitable for veterinary or human pharmaceutical use.

“Pharmaceutically acceptable salt” refers to a salt of a compound that is pharmaceutically acceptable and that possesses (or can be converted to a form that possesses) the desired pharmacological activity of the parent compound. Such salts include acid addition salts formed with inorganic acids such as hydrochloric acid, hydrobromic acid, sulfuric acid, nitric acid, phosphoric acid, and the like; or formed with organic acids such as acetic acid, benzenesulfonic acid, benzoic acid, camphorsulfonic acid, citric acid, ethanesulfonic acid, fumaric acid, glucoheptonic acid, gluconic acid, lactic acid, maleic acid, malonic acid, mandelic acid, methanesulfonic acid, 2-napththalenesulfonic acid, oleic acid, palmitic acid, propionic acid, stearic acid, succinic acid, tartaric acid, p-toluenesulfonic acid, trimethylacetic acid, and the like, and salts formed when an acidic proton present in the parent compound is replaced by either a metal ion, e.g., an alkali metal ion, an alkaline earth ion, or an aluminum ion; or coordinates with an organic base such as diethanolamine, triethanolamine, N-methylglucamine and the like. Also included in this definition are ammonium and substituted or quaternized ammonium salts. Representative non-limiting lists of pharmaceutically acceptable salts can be found in S. M. Berge et al., J. Pharma Sci., 66(1), 1-19 (1977), and Remington: The Science and Practice of Pharmacy, R. Hendrickson, ed., 21st edition, Lippincott, Williams & Wilkins, Philadelphia, Pa., (2005), at p. 732, Table 38-5, both of which are hereby incorporated by reference herein.

Provided are also compounds in which from 1 to n hydrogen atoms attached to a carbon atom may be replaced by a deuterium atom or D, in which n is the number of hydrogen atoms in the molecule. As known in the art, the deuterium atom is a non-radioactive isotope of the hydrogen atom. Such compounds exhibit may increase resistance to metabolism, and thus may be useful for increasing the half-life of the compounds when administered to a mammal. See, e.g., Foster, “Deuterium Isotope Effects in Studies of Drug Metabolism”, Trends Pharmacol. Sci., 5(12):524-527 (1984). Such compounds are synthesized by means well known in the art, for example by employing starting materials in which one or more hydrogen atoms have been replaced by deuterium.

Also provided herein are isotopically labeled forms of compounds detailed herein. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶C; and ¹²⁵I. Various isotopically labeled compounds of the present disclosure, for example those into which radioactive isotopes such as ³H, ¹³C and ¹⁴C are incorporated, are provided. Such isotopically labeled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of subjects (e.g. humans). Also provided for isotopically labeled compounds described herein are any pharmaceutically acceptable salts, or hydrates, as the case may be.

In one embodiment, the compounds disclosed herein may be varied such that from 1 to n hydrogens attached to a carbon atom is/are replaced by deuterium, in which n is the number of hydrogens in the molecule. Such compounds may exhibit increased resistance to metabolism and are thus useful for increasing the half life of the compound when administered to a mammal. See, for example, Foster, “Deuterium Isotope Effects in Studies of Drug Metabolism”, Trends Pharmacol. Sci. 5(12):524-527 (1984). Such compounds are synthesized by means well known in the art, for example by employing starting materials in which one or more hydrogens have been replaced by deuterium.

Deuterium labeled or substituted therapeutic compounds of the disclosure may have improved DMPK (drug metabolism and pharmacokinetics) properties, relating to absorption, distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies. Isotopically labeled compounds of this disclosure can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in the compounds provided herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

“Effective amount” or “therapeutically effective amount” means the amount of a compound described herein that may be effective to elicit the desired biological or medical response. These terms include the amount of a compound that, when administered to a subject for treating a disease, is sufficient to effect such treatment for the disease. The effective amount will vary depending on the compound, the disease and its severity and the age, weight, etc., of the subject to be treated.

“Subject” and “subjects” refers to humans, domestic animals (e.g., dogs and cats), farm animals (e.g., cattle, horses, sheep, goats and pigs), laboratory animals (e.g., mice, rats, hamsters, guinea pigs, pigs, rabbits, dogs, and monkeys), and the like.

“Treating” and “treatment” of a disease include the following: (1) preventing or reducing the risk of developing the disease, i.e., causing the clinical symptoms of the disease not to develop in a subject that may be exposed to or predisposed to the disease but does not yet experience or display symptoms of the disease, (2) inhibiting the disease, i.e., arresting or reducing the development of the disease or its clinical symptoms, and (3) relieving the disease, i.e., causing regression of the disease or its clinical symptoms.

The methods described herein may be applied to cell populations in vivo or ex vivo. “In vivo” means within a living individual, as within an animal or human. In this context, the methods described herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. In this context, the compounds and compositions described herein may be used for a variety of purposes, including therapeutic and experimental purposes. For example, the compounds and compositions described herein may be used ex vivo to determine the optimal schedule and/or dosing of administration of a compound of the present disclosure for a given indication, cell type, individual, and other parameters. Information gleaned from such use may be used for experimental purposes or in the clinic to set protocols for in vivo treatment. Other ex vivo uses for which the compounds and compositions described herein may be suited are described below or will become apparent to those skilled in the art. The selected compounds may be further characterized to examine the safety or tolerance dosage in human or non-human subjects. Such properties may be examined using commonly known methods to those skilled in the art.

“The terms “synergy” and “synergistic effect” encompass a more than additive effect of two or more agents compared to their individual effects. In certain embodiments, synergy or synergistic effect refers to an advantageous effect of using two or more agents in combination, e.g., in a pharmaceutical composition, or in a method of treatment. A synergistic effect may be attained when the active ingredients are: (1) co-formulated and administered or delivered simultaneously in a combined formulation; (2) delivered by alternation or in parallel as separate formulations; or (3) by some other regimen. When delivered in alternation therapy, a synergistic effect may be attained when the compounds are administered or delivered sequentially, e.g. in separate tablets, pills or capsules, or by different injections in separate syringes. In general, during alternation therapy, an effective dosage of each active ingredient is administered sequentially, i.e. serially, whereas in combination therapy, effective dosages of two or more active ingredients are administered together. As used herein, the synergistic anti-cancer effect of the combination of a Bcl-2 inhibitor and a BET inhibitor is greater than the predicted purely additive effects of the individual compounds of the combination.

### Combination Therapies

It is discovered herein (see, e.g., Examples 1-4) that the combination of a Bcl-2 inhibitor and a BET inhibitor resulted in superior growth inhibition in certain cancer cell lines, demonstrating that such combinations provide broader and superior efficacies in cancer treatments. In accordance with one embodiment of the disclosure, therefore, provided is a composition that includes a Bcl-2 inhibitor and a BET inhibitor. Also provided, in one embodiment, is a method of treating cancer that entails administering to a human a Bcl-2 inhibitor and a BET inhibitor, which can be administered concurrently or sequentially in need thereof.

### Bcl-2 Inhibitors

The Bcl-2 inhibitor can be a small molecule or a biologic. In one embodiment, the Bcl-2 inhibitor is a Bcl-2 selective inhibitor. In one embodiment, a Bcl-2 inhibitor can be selected from a group consisting of ABT-199 (venetoclax), ABT-737, ABT-263 (navitoclax), AT-101 (Gossypol), apogossypol, TW-37, G3139 (Genasense or oblimersen), obatoclax, sabutoclax, HA14-1, antimycin A, and S44563. In one embodiment, a Bcl-2 inhibitor can be selected from a combination of members of the group consisting of ABT-199 (venetoclax), ABT-737, ABT-263 (navitoclax), AT-101 (Gossypol), apogossypol, TW-37, G3139 (Genasense or oblimersen), obatoclax, sabutoclax, HA14-1, antimycin A, and S44563. For example, the Bcl-2 inhibitor can be venetoclax (described in U.S. Pat. Pub. No.: 2010/0305122):

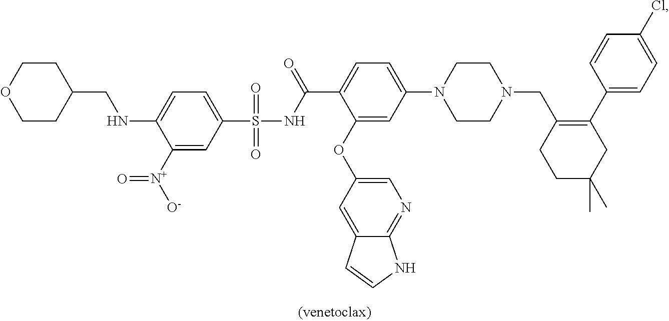 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

or a pharmaceutically acceptable salt, complex, solvate, prodrug, stereoisomer, mixture of stereoisomers or hydrate thereof.

In addition to the chemical structure, venetoclax may also be referred to or identified as (4-(4-({[2-(4-chlorophenyl)-4,4-dimethylcyclohex-1-en-1-yl]methyl}piperazin-1-yl)-N-({3-nitro-4-[(tetrahydro-2H-pyran-4-yl-methyl)amino]phenyl}sulfonyl)-2-(1H-pyrrolo[2,3-b]pyridin-5-yl-oxy)benzamide), 4-[4-[[2-(4-chlorophenyl)-4,4-dimethyl-1-cyclohexen-1-yl]methyl]-1-piperazinyl]-N-[[3-nitro-4-[[(tetrahydro-2H-pyran-4-yl)methyl]amino]phenyl]sulfonyl]-2-(1H-pyrrolo[2,3-h]pyridin-5-yloxy)-benzamide, ABT-199, GDC-0199, or RG7601.

Crystalline forms of venetoclax useful in the methods and combinations herein are disclosed in WO 2012/071336 (Catron et al.). In one embodiment, the crystalline forms of venetoclax can be free base anhydrate, free base hydrate, free base dichloromethane solvate, free base ethyl acetate solvate, free base acetonitrile solvate, free base acetone solvate, hydrochloride, free base hydrochloride, free base hydrochloride hydrate, free base sulfate, free base tetrahydrofuran, or combinations thereof.

Venetoclax (ABT-199) is a potent and selective Bcl-2 inhibitor shown to more potently inhibit cell growth and induce apoptosis in cell lines expressing high levels of Bcl-2. Navitoclax inhibits Bcl-2 but also targets Bcl-xL, which was shown to cause on-target thrombocytopenia. Unlike navitoclax, venetoclax avoids thrombocytopenia by specifically inhibiting Bcl-2 and sparing Bcl-xL.

In one embodiment, the Bcl-2 inhibitor can be ABT-737 (described in U.S. Pat. Pub. No.: 2007/0072860):

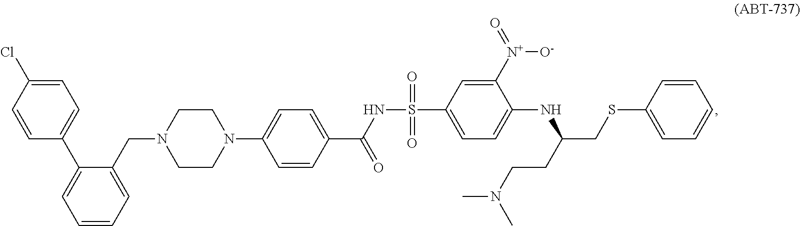 [SMILES: CN(C)CC[C@H](CSc1ccccc1)Nc1ccc(S(=O)(=O)NC(=O)c2ccc(N3CCN(Cc4ccccc4-c4ccc(Cl)cc4)CC3)cc2)cc1[N+](=O)[O-]]

or a pharmaceutically acceptable salt, complex, solvate, prodrug, stereoisomer, mixture of stereoisomers or hydrate thereof.

In addition to the chemical structure, ABT-737 may also be referred to or identified as 4-(4-((4′-chloro-[1,1′-biphenyl]-2-yl)methyl)piperazin-1-yl)-N-((4-((4-(dimethylamino)-1-(phenylthio)butan-2-yl)amino)-3-nitrophenyl)sulfonyl)benzamide, or 4-[4-[(4′-chloro[1,1′-biphenyl]-2-yl)methyl]-1-piperazinyl]-N-[[4-[[(1R)-3-(dimethylamino)-1-[(phenylthio)methyl]propyl]amino]-3-nitrophenyl]sulfonyl]-benzamide.

In one embodiment, the Bcl-2 inhibitor can be navitoclax (described in U.S. Pat. Pub. No.: 2007/0027135):

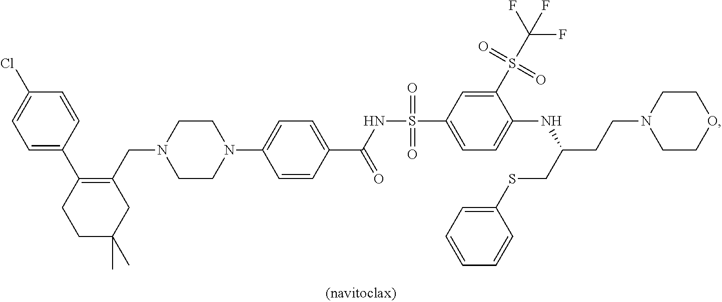 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

or a pharmaceutically acceptable salt, complex, solvate, prodrug, stereoisomer, mixture of stereoisomers or hydrate thereof.

In addition to the chemical structure, navitoclax may also be referred to or identified as (R)-4-(4-((4′-chloro-4,4-dimethyl-3,4,5,6-tetrahydro-[1,1′-biphenyl]-2-yl)methyl)piperazin-1-yl)-N-((4-((4-morpholino-1-(phenylthio)butan-2-yl)amino)-3-((trifluoromethyl)sulfonyl)phenyl)sulfonyl)benzamide, 4-(4-{[2-(4-chlorophenyl)-5,5-dimethyl-1-cyclohexen-1-yl]methyl}-1-piperazinyl)-N-[(4-{[(2R)-4-(4-morpholinyl)-1-(phenylsulfanyl)-2-butanyl]amino}-3-[(trifluoromethyl)sulfonyl]phenyl)sulfonyl]benzamide, or ABT-263.

In one embodiment, navitoclax is used as the navitoclax bis-HCl salt, as described in U.S. Pub. No.: 2010/0305125 (Borchardt, et al.). In one embodiment, navitoclax is utilized in the crystalline forms taught by U.S. Pub. No.: 2011/0071151 (Zhang et al.). In one embodiment, navitoclax is used as navitoclax free base in a solid crystalline form (e.g., Form I, or Form II), as taught by U.S. Pub. No.: 2011/0071151 (Zhang et al.).

In one embodiment, the Bcl-2 inhibitor can be AT-101:

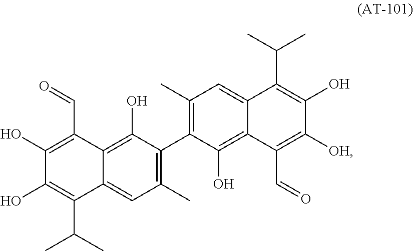 [SMILES: Cc1cc2c(C(C)C)c(O)c(O)c(C=O)c2c(O)c1-c1c(C)cc2c(C(C)C)c(O)c(O)c(C=O)c2c1O]

or a pharmaceutically acceptable salt, complex, solvate, prodrug, stereoisomer, mixture of stereoisomers or hydrate thereof.

In addition to the chemical structure, AT-101 may also be referred to or identified as (−)-1,1′,6,6′,7,7′-Hexahydroxy-3,3′-dimethyl-5,5′-bis(1-methylethyl)-[2,2′-binaphthalene]-8,8′-dicarboxaldehyde, (R)-(−)-gossypol, (R)-1,1′,6,6′,7,7′-Hexahydroxy-3,3′-dimethyl-5,5′-bis(1-methylethyl)-[2,2′-binaphthalene]-8,8′-dicarboxaldehyde, (R)-Gossypol, or AT 101.

In one embodiment, the Bcl-2 inhibitor can be apogossypol:

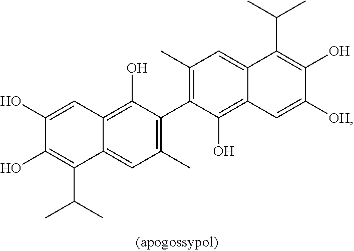 [SMILES: Cc1cc2c(C(C)C)c(O)c(O)cc2c(O)c1-c1c(C)cc2c(C(C)C)c(O)c(O)cc2c1O]

or a pharmaceutically acceptable salt, complex, solvate, prodrug, stereoisomer, mixture of stereoisomers or hydrate thereof.

In addition to the chemical structure, apogossypol may also be referred to or identified as 3-methyl-5-propan-2-yl-2-(1,6,7-trihydroxy-3-methyl-5-propan-2-ylnaphthalen-2-yl)naphthalene-1,6,7-triol, 5,5′-Diisopropyl-3,3′-dimethyl-[2,2′-binaphthalene]-1,1′,6,6′,7,7′-hexaol, NSC-736630, (2,2′-Binaphthalene)-1, 1′,6,6′,7,7′-hexol, 5,5′-diisopropyl-3,3′-dimethyl-, or 475-56-9.

In one embodiment, the Bcl-2 inhibitor can be TW-37:

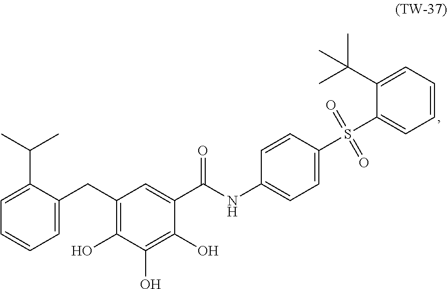 [SMILES: CC(C)c1ccccc1Cc1cc(C(=O)Nc2ccc(S(=O)(=O)c3ccccc3C(C)(C)C)cc2)c(O)c(O)c1O]

or a pharmaceutically acceptable salt, complex, solvate, prodrug, stereoisomer, mixture of stereoisomers or hydrate thereof.

In addition to the chemical structure, TW-37 may also be referred to or identified as N-{4-[(2-tert-butylphenyl)sulfonyl]phenyl}-2,3,4-trihydroxy-5-(2-isopropylbenzyl)benzamide, 877877-35-5, C33H35NO6S, CHEMBL217354, TW37, TW 37, or 877877-35-5.

In one embodiment, the Bcl-2 inhibitor can be obatoclax:

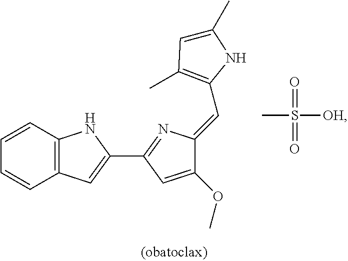

or a pharmaceutically acceptable salt, complex, solvate, prodrug, stereoisomer, mixture of stereoisomers or hydrate thereof.

In addition to the chemical structure, obatoclax may also be referred to or identified as (2Z)-2-[(5Z)-5-[(3,5-dimethyl-1H-pyrrol-2-yl)methylidene]-4-methoxypyrrol-2-ylidene]indole, 803712-79-0, Obatoclax mesylate, 2-(2-((3,5-Dimethyl-1H-pyrrol-2-yl)methylene)-3-methoxy-2H-pyrrol-5-yl)-1H-indole methanesulfonate, obatoclax mesylate, or GX15-070MS.

In one embodiment, the Bcl-2 inhibitor can be sabutoclax:

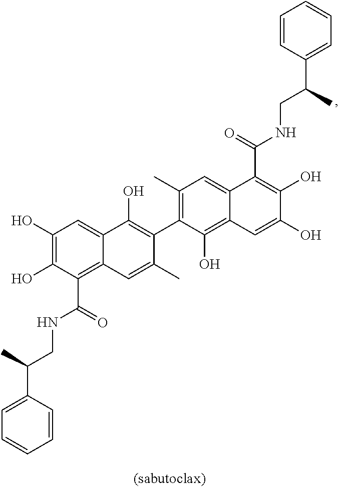 [SMILES: Cc1cc2c(C(=O)NC[C@H](C)c3ccccc3)c(O)c(O)cc2c(O)c1-c1c(C)cc2c(C(=O)NC[C@H](C)c3ccccc3)c(O)c(O)cc2c1O]

or a pharmaceutically acceptable salt, complex, solvate, prodrug, stereoisomer, mixture of stereoisomers or hydrate thereof.

In addition to the chemical structure, sabutoclax may also be referred to or identified as 2,3,5-trihydroxy-7-methyl-N-[(2R)-2-phenylpropyl]-6-[1,6,7-trihydroxy-3-methyl-5-[[(2R)-2-phenylpropyl]carbamoyl]naphthalen-2-yl]naphthalene-1-carboxamide, 1228108-65-3, UNII-39Y89ZRK34, GTPL7920, or CHEMBL1094250.

In one embodiment, the Bcl-2 inhibitor can be HA14-1:

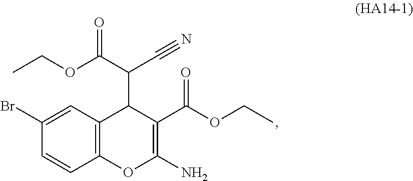 [SMILES: CCOC(=O)C1=C(N)Oc2ccc(Br)cc2C1C(C#N)C(=O)OCC]

or a pharmaceutically acceptable salt, complex, solvate, prodrug, stereoisomer, mixture of stereoisomers or hydrate thereof.

In addition to the chemical structure, HA14-1 may also be referred to or identified as ethyl 2-amino-6-bromo-4-(1-cyano-2-ethoxy-2-oxoethyl)-4H-chromene-3-carboxylate, 65673-63-4, ethyl 2-amino-6-bromo-4-(1-cyano-2-ethoxy-2-oxoethyl)-4H-chromene-3-carboxylate, HA 14-1, or ethyl [2-amino-6-bromo-4-(1-cyano-2-ethoxy-2-oxoethyl)]-4H-chromene-3-carboxylate.

In one embodiment, the Bcl-2 inhibitor can be antimycin A:

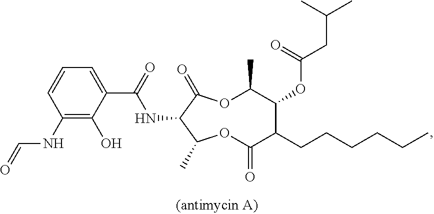 [SMILES: CCCCCCC1C(=O)O[C@H](C)[C@H](NC(=O)c2cccc(NC=O)c2O)C(=O)O[C@@H](C)[C@@H]1OC(=O)CC(C)C]

or a pharmaceutically acceptable salt, complex, solvate, prodrug, stereoisomer, mixture of stereoisomers or hydrate thereof.

In addition to the chemical structure, antimycin A may also be referred to or identified as [(2R,3S,6S,7R)-3-[(3-formamido-2-hydroxybenzoyl)amino]-8-hexyl-2,6-dimethyl-4,9-di oxo-1,5-dioxonan-7-yl] 3-methylbutanoate, 1397-94-0, or A8674_SIGMA.

### BET Inhibitors

The BET family includes BRD2, BRD3, BRD4, which are widely expressed across diverse tissues, and BRDT, which is expressed in the testes. The BET proteins regulate specific gene transcription by recognizing acetylated lysine residues within histone proteins at target genes and in turn recruiting factors that regulate RNA polymerase II activity. Each BET family member contains two highly conserved bromodomain motifs arranged in tandem, which specifically bind acetylated lysine residues on the amino-terminal tail of histones H3 and H4. BRD2, BRD3 and BRD4 regulate the transcription of key oncogenes, including MYC, resulting in cancer cell growth inhibition.

In one embodiment, BET inhibitors bind the bromodomains of BET proteins BRD2, BRD3, BRD4, and BRDT, and prevent protein-protein interaction between BET proteins and acetylated histones and transcription factors. In one embodiment, a BET inhibitor include a modulator of bromodomain-containing proteins. In one embodiment, the BET inhibitor is an inhibitor of bromodomain-containing protein 2 (BRD2), BRD3, BRD4, and/or BRDT.

In one embodiment, the BET inhibitor is a compound of Formula (I):

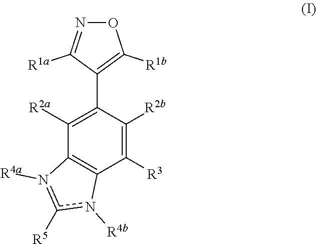

wherein
one [symbol] is a single bond and the other [symbol] is a double bond;
R^{1a} and R^{1b} are each independently C₁₋₆ alkyl optionally substituted with from 1 to 5 R²⁰ groups;
R^{2a} and R^{2b} are each independently H or halo;
R³ is —B(OH)₂, —B(OR^{a})₂, halo, —C(O)OR^{a}, —NHC(O)OR^{a}, —NHS(O)₂R^{a}, —S(O)₂NR^{a}R^{b}, C₁₋₁₀ alkyl, C₁₋₁₀ alkoxy, amino, C₅₋₁₀ aryl, C₆₋₂₀ arylalkyl, C₁₋₁₀ heteroalkyl, C₅₋₁₀ heteroaryl, or C₆₋₂₀ heteroarylalkyl,
wherein each of C₁₋₁₀ alkyl, C₁₋₁₀alkoxy, amino, C₅₋₁₀ aryl, C₆₋₂₀ arylalkyl, C₁₋₁₀ heteroalkyl, C₅₋₁₀ heteroaryl, or C₆₋₂₀ heteroarylalkyl is optionally substituted with from 1 to 5 R²⁰ groups;
   one of R^{4a} and R^{4b} is selected from the group consisting of H and C₁₋₆ alkyl optionally substituted with from 1 to 5 R² groups, and the other is absent;
   R⁵ is —C(O)OR^{a}, —NHC(O)OR^{a}, —NHS(O)₂R, or —S(O)₂NR^{a}R^{b}, H, C₁₋₁₀ alkyl, C₁₋₁₀ haloalkyl, C₁₋₁₀ alkoxy, amino, C₅₋₁₀ aryl, C₆₋₂₀ arylalkyl, C₁₋₁₀ heteroalkyl, C₅₋₁₀ heteroaryl, or C₆₋₂₀ heteroarylalkyl,
wherein each of C₁₋₁₀ alkyl, C₁₋₁₀ haloalkyl, C₁₋₁₀ alkoxy, amino, C₅₋₁₀ aryl, C₆₋₂₀ arylalkyl, C₁₋₁₀ heteroalkyl, C₅₋₁₀ heteroaryl, and C₆₋₂₀ heteroarylalkyl is optionally substituted with from 1 to 5 R²⁰ groups;
   each R^{a} and R^{b} is independently selected from the group consisting of H, C₁₋₁₀ alkyl, C₅₋₁₀ aryl, C₆₋₂₀ arylalkyl, C₁₋₁₀ heteroalkyl, C₁₋₁₀ heteroaryl, and C₆₋₂₀ heteroarylalkyl, each of which is optionally substituted with from 1 to 5 R²⁰ groups; and
   each R²⁰ is independently selected from the group consisting of acyl, C₁₋₁₀ alkyl, C₁₋₁₀ alkoxy, amino, amido, amidino, C₅₋₁₀ aryl, C₆₋₂₀ arylalkyl, azido, carbamoyl, carboxyl, carboxyl ester, cyano, guanidino, halo, C₁₋₁₀ haloalkyl, C₁₋₁₀ heteroalkyl, C₅₋₁₀ heteroaryl, C₆₋₂₀ heteroarylalkyl, hydroxy, hydrazino, imino, oxo, nitro, sulfinyl, sulfonic acid, sulfonyl, thiocyanate, thiol, and thione,
   or a pharmaceutically acceptable salt, complex, solvate, prodrug, stereoisomer, mixture of stereoisomers or hydrate thereof.

Compounds of Formula (I) (which include compounds of any of Formulae (Ia), (Ib), (Ic), (Id) and (Ie), described below) can include, independently, one or more of the following features. It will be recognized that features specified in each embodiment may be combined with other specified features to provide further embodiments.

In one embodiment, R^{1a} and R^{1b} are each independently C₁₋₆ alkyl. In one embodiment, R^{1a} and R^{1b} are different, and in other compounds R^{1a} and R^{1b} are the same. In some compounds, R^{1a} and R^{1b} are each independently a C₁₋₆ alkyl optionally substituted with 1-5 R²⁰ groups. In one embodiment, R^{1a} and R^{1b} are both methyl. In one embodiment, one of R^{1a} and R^{1b} is a methyl and the other is a methyl substituted with a hydroxy. In one embodiment, R^{1a} and R^{1b} are both methyl substituted with a hydroxy. In one embodiment, one of R^{1a} and R^{1b} is a methyl and the other is a methyl substituted with an amine. In some compounds, R^{1a} and R^{1b} are both methyl substituted with an amine.

In one embodiment, R^{2a} and R^{2b} are both H. In one embodiment, R^{2a} and R^{2b} are both halo. In one embodiment, one of R^{2a} and R^{2b} is H and the other is halo. In one embodiment, the halo is —F or —Cl.

In one embodiment, R³ is —B(OH)₂, —B(OR^{a})₂, or halo. In some compounds, R³ is —C(O)OR^{a}, —NHC(O)OR^{a}, —NHS(O)₂R^{a}, or —S(O)₂NR^{a}R^{b} wherein R^{a} and R^{b} are described above. In some compounds, R³ is —C(O)OR^{a}, —NHC(O)OR^{a}, —NHS(O)₂R^{a}, or —S(O)₂NR^{a}R^{b}, wherein each R^{a} and R^{b} is independently C₁₋₁₀alkyl, C₅₋₁₀ aryl, C₁₋₁₀ heteroalkyl or C₅₋₁₀ heteroaryl, each of which may be optionally substituted as described above. For example, in one embodiment, R³ is —C(O)OR^{a}, —NHC(O)OR^{a}, —NHS(O)₂R^{a}, or —S(O)₂NR^{a}R^{b}, wherein each R^{a} and R^{b} is independently C₅₋₁₀ aryl or C₅₋₁₀ heteroaryl. In one embodiment, R³ is selected from the group consisting of C₁₋₁₀ alkyl, C₁₋₁₀ alkoxy, amino, C₅₋₁₀ aryl, C₆₋₂₀ arylalkyl, C₁₋₁₀ heteroalkyl, C₅₋₁₀ heteroaryl, and C₆₋₂₀ heteroarylalkyl, each of which is optionally substituted with from 1 to 5 R⁰ groups, wherein R²⁰ is described above. In one embodiment, R³ is C₁₋₁₀ alkyl, C₁₋₁₀alkoxy, or C₁₋₁₀ heteroalkyl, each of which may be optionally substituted as described above. In one embodiment, the heteroalkyl is a heterocycloalkyl. In other embodiments, R³ is C₆₋₂₀ arylalkyl or C₆₋₂₀ heteroarylalkyl, each of which may be optionally substituted as described above. In other embodiments, R³ is C₅₋₁₀ aryl, C₆₋₂₀ arylalkyl, C₅₋₁₀ heteroaryl, or C₆₋₂₀ heteroarylalkyl, each of which may be optionally substituted as described above. In one embodiment, R³ is amino optionally substituted as described above. For example, in one embodiment, R³ is —NH₂, and in other embodiments, R³ is —NR^{y}R^{z}, wherein R^{y} and R^{z} together with the nitrogen to which they are bonded form a C₁₋₁₀ heteroalkyl or C₅₋₁₀ heteroaryl, each of which may be optionally substituted as described above.

Other non-limiting examples of R³ include the following:

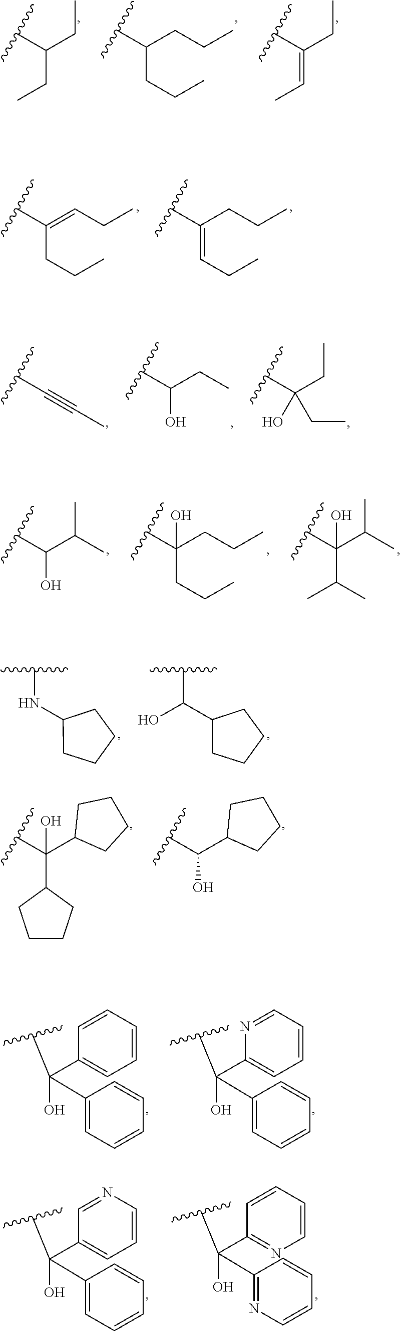

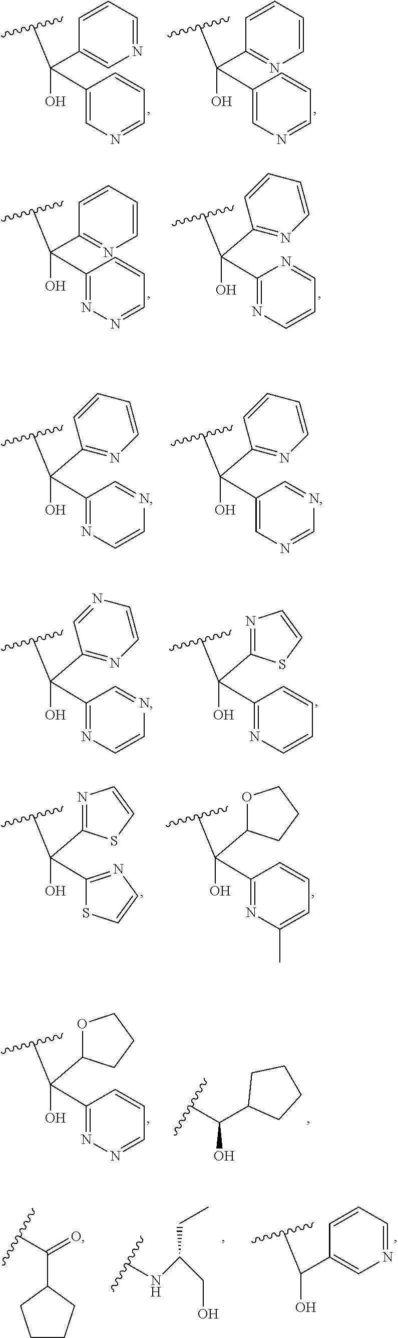

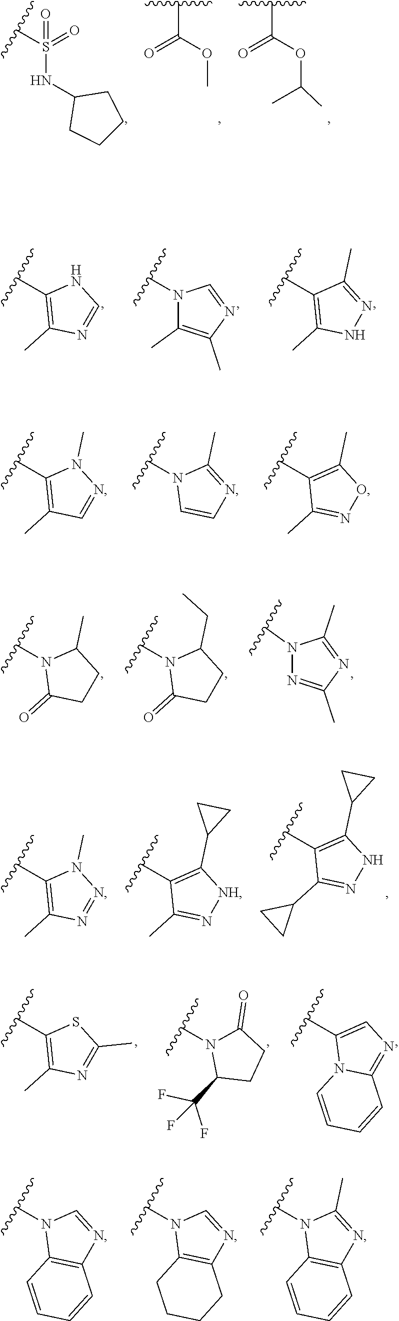

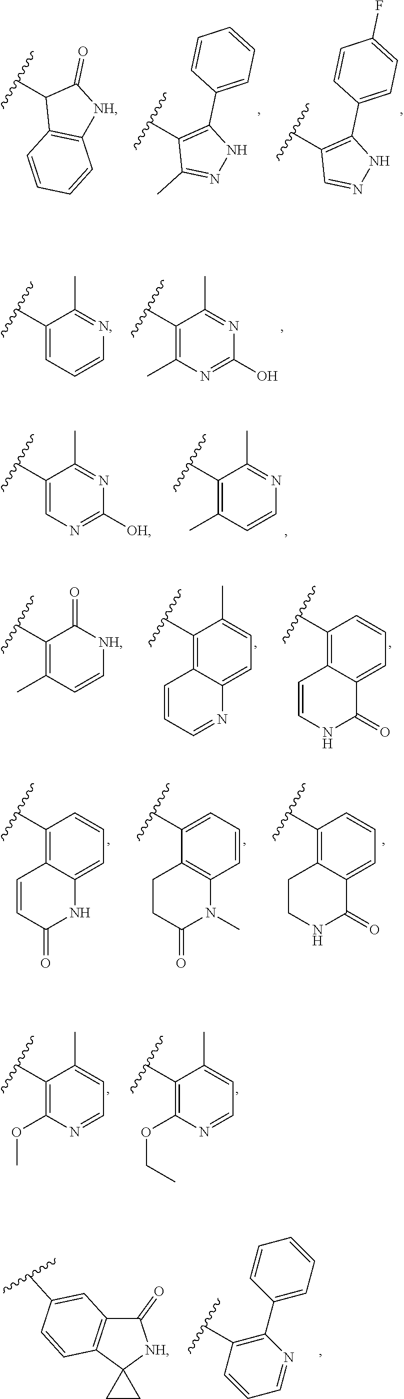

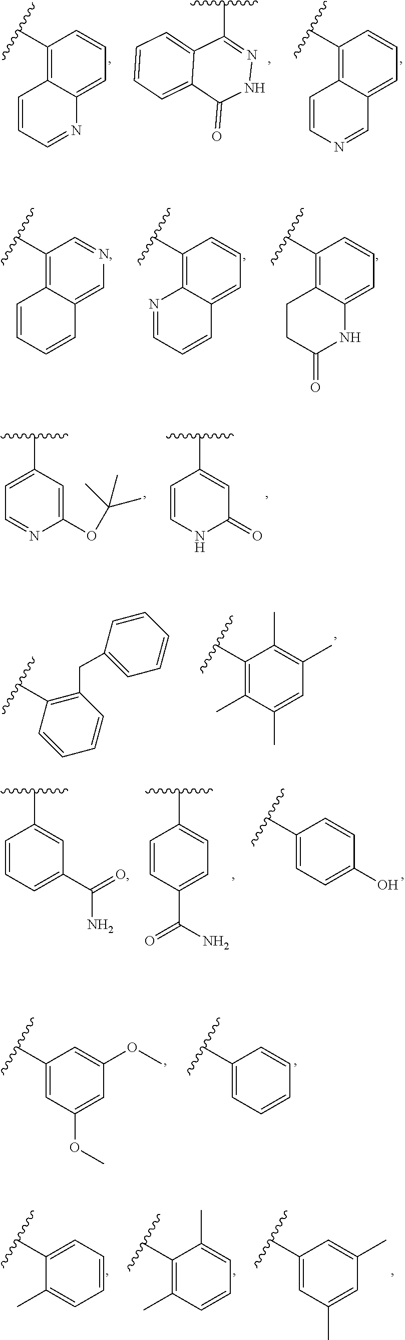

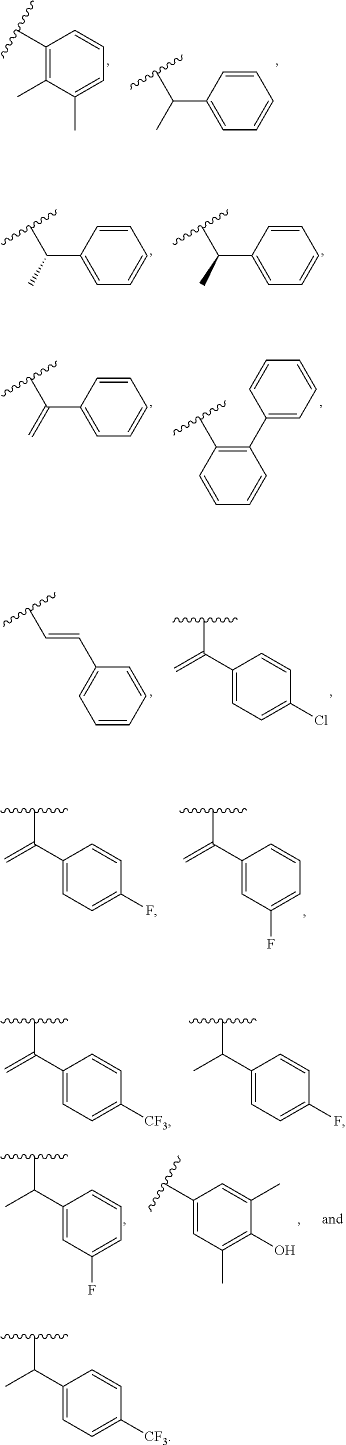

In one embodiment, one of R^{4a} or R^{4b} is H and the other is absent, that is, in some compounds R^{4a} is H and R^{4b} is absent, and in other compounds R^{4a} is absent and R^{4b} is H. In other embodiments, one of R^{4a} and R^{4b} is alkyl and the other is absent, that is, in some compounds R^{4a} is alkyl and R^{4b} is absent, and in other compounds R^{4a} is absent and R^{4b} is alkyl. In one embodiment, the alkyl is methyl.

In one embodiment, R⁵ is —C(O)OR^{a}, —NHC(O)OR^{a}, —NHS(O)₂R^{a}, or —S(O)₂NR^{a}R^{b}, wherein R^{a} and R^{b} are described above. In one embodiment, R⁵ is —C(O)OR^{a}, —NHC(O)OR^{a}, —NHS(O)₂R^{a}, or —S(O)₂NR^{a}R^{b}, wherein each R^{a} and R^{b} is independently C₁₋₁₀ alkyl or C₅₋₁₀ aryl, each of which may be optionally substituted as described above. For example, in one embodiment, R⁵ is —NHC(O)OR^{a}, wherein R^{a} is methyl. In one embodiment, R⁵ is —NHS(O)₂R^{a}, wherein R^{a} is C₁₋₁₀ alkyl or C₅₋₁₀ aryl, each of which may be optionally substituted as described above. For example, in one embodiment, R⁵ is —NHS(O)₂R^{a}, wherein R^{a} is cyclopropyl. In one embodiment, R⁵ is selected from the group consisting of H, C₁₋₁₀ alkyl, C₁₋₁₀ haloalkyl, C₁₋₁₀ alkoxy, amino, C₅₋₁₀ aryl, C₆₋₂₀ arylalkyl, C₁₋₁₀ heteroalkyl, C₁₋₁₀ heteroaryl, and C₆₋₂₀ heteroarylalkyl, each of which is optionally substituted with from 1 to 5 R²⁰ groups, wherein R²⁰ is described above. In one embodiment, R⁵ is C₁₋₁₀ alkyl optionally substituted as described above. In some compounds the C₁₋₁₀ alkyl is a C₁₋₁₀ cycloalkyl, e.g. cyclopropyl. In other embodiments, R⁵ is amino optionally substituted as described above. For example, in one embodiment, R⁵ is —NH₂, and in other embodiments, R⁵ is —NR^{y}R^{z}, wherein R^{y} is H and R^{z} is alkyl, e.g. cyclopropyl. In other embodiments, R⁵ is alkoxy, e.g. methoxy.

In one embodiment, R^{1a}, R^{1b}, R³, R^{4a}, R^{4b} and R⁵ are optionally substituted with from 1 to 5 (i.e. 1, 2, 3, 4 or 5) R² groups as described above. In one embodiment, R^{1a}, R^{1b}, R³, R^{4a}, R^{4b} and R⁵ are optionally substituted with 1, 2, or 3 R²⁰ groups. In one embodiment, each R²⁰ is independently selected from the group consisting of alkyl, alkoxy, amino, cyano, halo, haloalkyl, heteroalkyl, hydroxy, and sulfonyl. In one embodiment, each R²⁰ is independently selected from the group consisting of aryl, alkylaryl, heteroaryl, and heteroalkylaryl. In one embodiment, R^{1a}, R^{1b}, R³, R^{4a}, R^{4b} and R⁵ are not substituted. In some compounds, R²⁰ is not substituted.

One subset of compounds of Formula (I) relates to compounds of Formula (Ia):

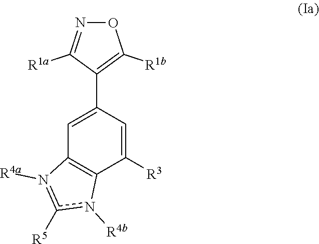

wherein [symbol]
is a single bond or a double bond;
R^{1a} and R^{1b} are each independently C₁₋₆ alkyl optionally substituted with from 1 to 5 R²⁰ groups;
R³ is boronic acid, boronic acid ester, halo, —C(O)OR^{a}, —NHC(O)OR^{a}, —NHS(O)₂R^{a}, —S(O)₂NR^{a}R^{b}, C₁₋₁₀alkyl, C₁₋₁₀ alkoxy, amino, C₅₋₁₀ aryl, C₆₋₂₀ arylalkyl, C₁₋₁₀ heteroalkyl, C₅₋₁₀ heteroaryl, or C₆₋₂₀ heteroarylalkyl,
wherein each of C₁₋₁₀ alkyl, C₁₋₁₀ alkoxy, amino, C₅₋₁₀ aryl, C₆₋₂₀ arylalkyl, C₁₋₁₀ heteroalkyl, C₅₋₁₀ heteroaryl, or C₆₋₂₀ heteroarylalkyl is optionally substituted with from 1 to 5 R²⁰ groups;
   one of R^{4a} and R^{4b} is selected from the group consisting of H and C₁₋₆ alkyl optionally substituted with from 1 to 5 R² groups, and the other is absent;
   R is —C(O)OR^{a}, —NHC(O)OR, —NHS(O)₂R^{a}, or —S(O)₂NR^{a}R^{b}, H, C₁₋₁₀ alkyl, C₁₋₁₀ haloalkyl, C₁₋₁₀ alkoxy, amino, C₅₋₁₀ aryl, C₆₋₂₀ arylalkyl, C₁₋₁₀ heteroalkyl, C₅₋₁₀ heteroaryl, or C₆₋₂₀ heteroarylalkyl,
wherein each of C₁₋₁₀ alkyl, C₁₋₁₀ haloalkyl, C₁₋₁₀ alkoxy, amino, C₅₋₁₀ aryl, C₆₋₂₀ arylalkyl, C₁₋₁₀ heteroalkyl, C₅₋₁₀ heteroaryl, and C₆₋₂₀ heteroarylalkyl is optionally substituted with from 1 to 5 R²⁰ groups;
   each R^{a} and R^{b} is independently selected from the group consisting of H, C₁₋₁₀ alkyl, C₅₋₁₀ aryl, C₆₋₂₀ arylalkyl, C₁₋₁₀ heteroalkyl, C₅₋₁₀ heteroaryl, and C₆₋₂₀ heteroarylalkyl, each of which is optionally substituted with from 1 to 5 R²⁰ groups; and
   each R²⁰ is independently selected from the group consisting of acyl, C₁₋₁₀ alkyl, C₁₋₁₀ alkoxy, amino, amido, amidino, C₅₋₁₀ aryl, C₆₋₂₀ arylalkyl, azido, carbamoyl, carboxyl, carboxyl ester, cyano, guanidino, halo, C₁₋₁₀ haloalkyl, C₁₋₁₀ heteroalkyl, C₁₋₁₀ heteroaryl, C₆₋₂₀ heteroarylalkyl, hydroxy, hydrazino, imino, oxo, nitro, sulfinyl, sulfonic acid, sulfonyl, thiocyanate, thiol, and thione,
   or a pharmaceutically acceptable salt, complex, solvate, prodrug, stereoisomer, mixture of stereoisomers or hydrate thereof.

Another subset of compounds of Formula (I) relates to compounds of Formula (Ib):

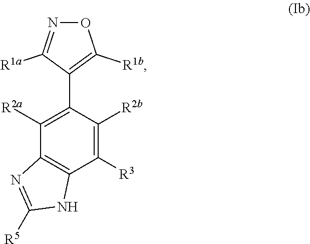

wherein:
R^{1a} and R^{1b} are each independently C₁₋₆ alkyl optionally substituted with from 1 to 5 R²⁰ groups;
R³ is —B(OH)₂, —B(OR^{a})₂, halo, —C(O)OR^{a}, —NHC(O)OR^{a}, —NHS(O)₂R^{a}, —S(O)₂NR^{a}R^{b}, C₁₋₁₀ alkyl, C₁₋₁₀ alkoxy, amino, C₅₋₁₀ aryl, C₆₋₂₀ arylalkyl, C₁₋₁₀ heteroalkyl, C₅₋₁₀ heteroaryl, or C₆₋₂₀ heteroarylalkyl,
wherein each of C₁₋₁₀ alkyl, C₁₋₁₀ alkoxy, amino, C₅₋₁₀ aryl, C₆₋₂₀ arylalkyl, C₁₋₁₀ heteroalkyl, C₅₋₁₀ heteroaryl, or C₆₋₂₀ heteroarylalkyl is optionally substituted with from 1 to 5 R² groups;
   R⁵ is —C(O)OR^{a}, —NHC(O)OR^{a}, —NHS(O)₂R^{a}, or —S(O)₂NR^{a}R^{b}, H, C₁₋₁₀ alkyl, C₁₋₁₀ haloalkyl, C₁₋₁₀ alkoxy, amino, C₅₋₁₀ aryl, C₆₋₂₀ arylalkyl, C₁₋₁₀ heteroalkyl, C₅₋₁₀ heteroaryl, or C₆₋₂₀ heteroarylalkyl,
wherein each of C₁₋₁₀ alkyl, C₁₋₁₀ haloalkyl, C₁₋₁₀ alkoxy, amino, C₅₋₁₀ aryl, C₆₋₂₀ arylalkyl, C₁₋₁₀ heteroalkyl, C₅₋₁₀ heteroaryl, and C₆₋₂₀ heteroarylalkyl is optionally substituted with from 1 to 5 R²⁰ groups;
   each R^{a} and R^{b} is independently selected from the group consisting of H, C₁₋₁₀ alkyl, C₅₋₁₀ aryl, C₆₋₂₀ arylalkyl, C₁₋₁₀ heteroalkyl, C₅₋₁₀ heteroaryl, and C₆₋₂₀ heteroarylalkyl, each of which is optionally substituted with from 1 to 5 R²⁰ groups; and
   each R²⁰ is independently selected from the group consisting of acyl, C₁₋₁₀ alkyl, C₁₋₁₀ alkoxy, amino, amido, amidino, C₅₋₁₀ aryl, C₆₋₂₀ arylalkyl, azido, carbamoyl, carboxyl, carboxyl ester, cyano, guanidino, halo, C₁₋₁₀ haloalkyl, C₁₋₁₀ heteroalkyl, C₅₋₁₀ heteroaryl, C₆₋₂₀ heteroarylalkyl, hydroxy, hydrazino, imino, oxo, nitro, sulfinyl, sulfonic acid, sulfonyl, thiocyanate, thiol, and thione,
   or a pharmaceutically acceptable salt, complex, solvate, prodrug, stereoisomer, mixture of stereoisomers or hydrate thereof.

Another subset of compounds of Formula (I) relates to compounds of Formula (Ic):

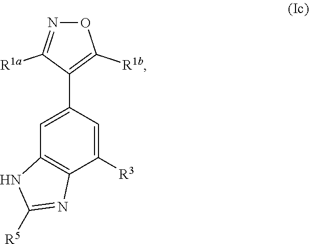

wherein:
R^{1a} and R^{1b} are each independently C₁₋₆ alkyl optionally substituted with from 1 to 5 R²⁰ groups;
R³ is —B(OH)₂, —B(OR^{a})₂, halo, —C(O)OR^{a}, —NHC(O)OR^{a}, —NHS(O)₂R^{a}, —S(O)₂NR^{a}R^{b}, C₁₋₁₀ alkyl, C₁₋₁₀ alkoxy, amino, C₅₋₁₀ aryl, C₆₋₂₀ arylalkyl, C₁₋₁₀ heteroalkyl, C₅₋₁₀ heteroaryl, or C₆₋₂₀ heteroarylalkyl,
wherein each of C₁₋₁₀ alkyl, C₁₋₁₀alkoxy, amino, C₅₋₁₀ aryl, C₆₋₂₀ arylalkyl, C₁₋₁₀ heteroalkyl, C₅₋₁₀ heteroaryl, or C₆₋₂₀ heteroarylalkyl is optionally substituted with from 1 to 5 R²⁰ groups;
   R⁵ is —C(O)OR^{a}, —NHC(O)OR^{a}, —NHS(O)₂R^{a}, or —S(O)₂NR^{a}R^{b}, H, C₁₋₁₀ alkyl, C₁₋₁₀ haloalkyl, C₁₋₁₀alkoxy, amino, C₅₋₁₀ aryl, C₆₋₂₀ arylalkyl, C₁₋₁₀ heteroalkyl, C₅₋₁₀ heteroaryl, or C₆₋₂₀ heteroarylalkyl,
wherein each of C₁₋₁₀ alkyl, C₁₋₁₀ haloalkyl, C₁₋₁₀ alkoxy, amino, C₅₋₁₀ aryl, C₆₋₂₀ arylalkyl, C₁₋₁₀ heteroalkyl, C₅₋₁₀ heteroaryl, and C₆₋₂₀ heteroarylalkyl is optionally substituted with from 1 to 5 R²⁰ groups;
   each R^{a} and R^{b} is independently selected from the group consisting of H, C₁₋₁₀ alkyl, C₅₋₁₀ aryl, C₆₋₂₀ arylalkyl, C₁₋₁₀ heteroalkyl, C₁₋₁₀ heteroaryl, and C₆₋₂₀ heteroarylalkyl, each of which is optionally substituted with from 1 to 5 R²⁰ groups; and
   each R²⁰ is independently selected from the group consisting of acyl, C₁₋₁₀ alkyl, C₁₋₁₀ alkoxy, amino, amido, amidino, C₅₋₁₀ aryl, C₆₋₂₀ arylalkyl, azido, carbamoyl, carboxyl, carboxyl ester, cyano, guanidino, halo, C₁₋₁₀ haloalkyl, C₁₋₁₀ heteroalkyl, C₅₋₁₀ heteroaryl, C₆₋₂₀ heteroarylalkyl, hydroxy, hydrazino, imino, oxo, nitro, sulfinyl, sulfonic acid, sulfonyl, thiocyanate, thiol, and thione,
   or a pharmaceutically acceptable salt, complex, solvate, prodrug, stereoisomer, mixture of stereoisomers or hydrate thereof.

Another subset of compounds of Formula (I) relates to compounds of Formula (Id):

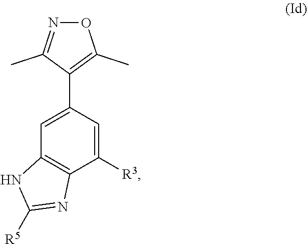

wherein:
R³ is —B(OH)₂, —B(OR^{a})₂, halo, —C(O)OR^{a}, —NHC(O)OR^{a}, —NHS(O)₂R^{a}, —S(O)₂NR^{a}R^{b}, C₁₋₁₀alkyl, C₁₋₁₀alkoxy, amino, C₅₋₁₀ aryl, C₆₋₂₀ arylalkyl, C₁₋₁₀ heteroalkyl, C₅₋₁₀ heteroaryl, or C₆₋₂₀ heteroarylalkyl,
wherein each of C₁₋₁₀ alkyl, C₁₋₁₀ alkoxy, amino, C₅₋₁₀ aryl, C₆₋₂₀ arylalkyl, C₁₋₁₀ heteroalkyl, C₅₋₁₀ heteroaryl, or C₆₋₂₀ heteroarylalkyl is optionally substituted with from 1 to 5 R²⁰ groups;
   R⁵ is —C(O)OR^{a}, —NHC(O)OR^{a}, —NHS(O)₂R^{a}, or —S(O)₂NR^{a}R^{b}, H, C₁₋₁₀ alkyl, C₁₋₁₀ haloalkyl, C₁₋₁₀ alkoxy, amino, C₅₋₁₀ aryl, C₆₋₂₀ arylalkyl, C₁₋₁₀ heteroalkyl, C₅₋₁₀ heteroaryl, or C₆₋₂₀ heteroarylalkyl,
wherein each of C₁₋₁₀alkyl, C₁₋₁₀ haloalkyl, C₁₋₁₀ alkoxy, amino, C₅₋₁₀ aryl, C₆₋₂₀ arylalkyl, C₁₋₁₀ heteroalkyl, C₅₋₁₀ heteroaryl, and C₆₋₂₀ heteroarylalkyl is optionally substituted with from 1 to 5 R²⁰ groups;
   each R^{a} and R^{b} is independently selected from the group consisting of H, C₁₋₁₀ alkyl, C₅₋₁₀ aryl, C₆₋₂₀ arylalkyl, C₁₋₁₀ heteroalkyl, C₅₋₁₀ heteroaryl, and C₆₋₂₀ heteroarylalkyl, each of which is optionally substituted with from 1 to 5 R²⁰ groups; and
   each R²⁰ is independently selected from the group consisting of acyl, C₁₋₁₀ alkyl, C₁₋₁₀ alkoxy, amino, amido, amidino, C₅₋₁₀ aryl, C₆₋₂₀ arylalkyl, azido, carbamoyl, carboxyl, carboxyl ester, cyano, guanidino, halo, C₁₋₁₀haloalkyl, C₁₋₁₀ heteroalkyl, C₁₋₁₀ heteroaryl, C₆₋₂₀ heteroarylalkyl, hydroxy, hydrazino, imino, oxo, nitro, sulfinyl, sulfonic acid, sulfonyl, thiocyanate, thiol, and thione,
   or a pharmaceutically acceptable salt, complex, solvate, prodrug, stereoisomer, mixture of stereoisomers or hydrate thereof.

Another subset of compounds of Formula (I) relates to compounds of Formula (Ie):

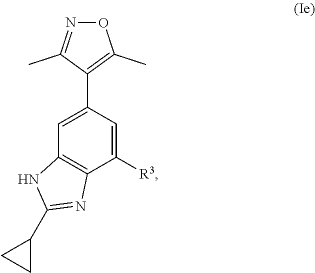

wherein:
R³ is —B(OH)₂, —B(OR^{a})₂, halo, —C(O)OR^{a}, —NHC(O)OR^{a}, —NHS(O)₂R^{a}, —S(O)₂NR^{a}R^{b}, C₁₋₁₀ alkyl, C₁₋₁₀ alkoxy, amino, C₅₋₁₀ aryl, C₆₋₂₀ arylalkyl, C₁₋₁₀ heteroalkyl, C₁₋₁₀ heteroaryl, or C₆₋₂₀ heteroarylalkyl,
wherein each of C₁₋₁₀ alkyl, C₁₋₁₀ alkoxy, amino, C₁₋₁₀ aryl, C₆₋₂₀ arylalkyl, C₁₋₁₀ heteroalkyl, C₅₋₁₀ heteroaryl, or C₆₋₂₀ heteroarylalkyl is optionally substituted with from 1 to 5 R²⁰ groups;
   each R^{a} and R^{b} is independently selected from the group consisting of H, C₁₋₁₀ alkyl, C₅₋₁₀ aryl, C₆₋₂₀ arylalkyl, C₁₋₁₀ heteroalkyl, C₁₋₁₀ heteroaryl, and C₆₋₂₀ heteroarylalkyl, each of which is optionally substituted with from 1 to 5 R²⁰ groups; and
   each R²⁰ is independently selected from the group consisting of acyl, C₁₋₁₀ alkyl, C₁₋₁₀ alkoxy, amino, amido, amidino, C₅₋₁₀ aryl, C₆₋₂₀ arylalkyl, azido, carbamoyl, carboxyl, carboxyl ester, cyano, guanidino, halo, C₁₋₁₀ haloalkyl, C₁₋₁₀ heteroalkyl, C₅₋₁₀ heteroaryl, C₆₋₂₀ heteroarylalkyl, hydroxy, hydrazino, imino, oxo, nitro, sulfinyl, sulfonic acid, sulfonyl, thiocyanate, thiol, and thione,
   or a pharmaceutically acceptable salt, complex, solvate, prodrug, stereoisomer, mixture of stereoisomers or hydrate thereof.

In separate embodiments within each of the compounds described for Formulas (I), (Ia), (Ib), and (Ic), there is one embodiment comprising a compound in which R^{1a} and R^{1b} are each independently C₁₋₆ alkyl.

In separate embodiments within each of the compounds described for Formulas (I), (la), (Ib), (Ic), (Id), and (Ie), there is one embodiment comprising a compound in which R³ is C₁₋₁₀ alkyl, C₁₋₁₀ alkoxy, or C₁₋₁₀ heteroalkyl, each of which may be optionally substituted with from 1 to 5 R²⁰ groups.

In separate embodiments within each of the compounds described for Formulas (I), (Ia), (Ib), (Ic), (Id), and (Ie), there is one embodiment comprising a compound in which R³ is C₅₋₁₀ aryl, C₆₋₂₀ arylalkyl, C₅₋₁₀ heteroaryl, or C₆₋₂₀ heteroarylalkyl, each of which may be optionally substituted with from 1 to 5 R²⁰ groups.

In separate embodiments within each of the compounds described for Formulas (I), (Ia), (Ib), (Ic), and (Id), there is one embodiment comprising a compound in which R⁵ is C₁₋₁₀ alkyl.

A separate embodiment comprises a compound of Formula (Ie), as defined above, wherein R³ is C₁₋₁₀ alkyl, C₁₋₁₀alkoxy, or C₁₋₁₀ heteroalkyl, each of which may be optionally substituted with from 1 to 5 R²⁰ groups.

There is also provided a separate embodiment with each of the embodiments described herein comprising a compound of Formula (Ie), further in which R³ is C₅₋₁₀ aryl, C₆₋₂₀ arylalkyl, C₁₋₁₀ heteroaryl, or C₆₋₂₀ heteroarylalkyl, each of which may be optionally substituted with from 1 to 5 R²⁰ groups.

The compounds which are BET inhibitors described above, including Compound A, may be prepared by methods disclosed in U.S. Pub. No.: 2014/0336190 (Gilead Sciences, Inc.).

### Treatment Methods and Uses

The Bcl-2 inhibitors and BET inhibitors described herein may be used in combination therapy. Accordingly, provided herein is a method for treating cancer in a human in need thereof, comprising administering to the human a therapeutically effective amount of a Bcl-2 inhibitor and a therapeutically effective amount of a BET inhibitor, as disclosed herein.

The present disclosure, in one embodiment, provides a method for treating cancer in a human in need thereof, comprising administering to the human a therapeutically effective amount of a Bcl-2 inhibitor and a therapeutically effective amount of a BET inhibitor, wherein the BET inhibitor is a compound of the Formula (I) as disclosed herein.

In one embodiment, the amount or dosage of the Bcl-2 inhibitor, the BET inhibitor, or both, used in combination, does not exceed the level at which each agent is used individually, e.g., as a monotherapy. In one embodiment, the amount or dosage of the Bcl-2 inhibitor, the BET inhibitor, or both, used in combination, is lower (e.g., at least 20%, at least 30%, at least 40%, or at least 50% lower) than the amount or dosage of each agent used individually, e.g., as a monotherapy. In one embodiment, the amount or dosage of the Bcl-2 inhibitor, the BET inhibitor, or both, used in combination that results in treatment of cancer is lower (e.g., at least 20%, at least 30%, at least 40%, or at least 50% lower) than the amount or dosage of each agent used individually, e.g., as a monotherapy.

In one embodiment, venetoclax is administered orally. In one embodiment, Compound A is administered orally. In various embodiments, venetoclax is administered prior to, after, or concurrently with Compound A.

The present disclosure, in one embodiment, provides a method for treating cancer in a human in need thereof, comprising administering to the human (i) a therapeutically effective amount of a Bcl-2 inhibitor, and (ii) a therapeutically effective amount of Compound A. In one embodiment, provided is a method of treating cancer in a human in need thereof, comprising administering to the human (i) a therapeutically effective amount of venetoclax, and (ii) a therapeutically effective amount of a BET inhibitor. In one embodiment, provided is a method treating cancer in a human in need thereof, comprising administering to the human (i) a therapeutically effective amount of venetoclax, and (ii) a therapeutically effective amount of Compound A.

Cancer

In various embodiments, the cancer is carcinoma, sarcoma, melanoma, lymphoma or leukemia. In one embodiment, the cancer is a hematologic malignancy. In one embodiment, the cancer is lymphoma (e.g., non-Hodgkin's lymphoma). In one embodiment, the cancer is leukemia (e.g., chronic lymphocytic leukemia). In one embodiment, the cancer is multiple myeloma. In one embodiment, the cancer is diffuse large B-cell lymphoma (DLBCL). In one embodiment, the cancer is follicular lymphoma (FL).

In various embodiments, the cancer is small lymphocytic lymphoma, non-Hodgkin's lymphoma, indolent non-Hodgkin's lymphoma (iNHL), refractory iNHL, mantle cell lymphoma, follicular lymphoma (FL), lymphoplasmacytic lymphoma, marginal zone lymphoma, immunoblastic large cell lymphoma, lymphoblastic lymphoma, Splenic marginal zone B-cell lymphoma (+/−villous lymphocytes), nodal marginal zone lymphoma (+/−monocytoid B-cells), extranodal marginal zone B-cell lymphoma of mucosa-associated lymphoid tissue type, cutaneous T-cell lymphoma, extranodal T-cell lymphoma, anaplastic large cell lymphoma, angioimmunoblastic T-cell lymphoma, mycosis fungoides, B-cell lymphoma, diffuse large B-cell lymphoma, mediastinal large B-cell lymphoma, intravascular large B-cell lymphoma, primary effusion lymphoma, small non-cleaved cell lymphoma, Burkitt's lymphoma, multiple myeloma, plasmacytoma, acute lymphocytic leukemia, T-cell acute lymphoblastic leukemia, B-cell acute lymphoblastic leukemia, B-cell prolymphocytic leukemia, acute myeloid leukemia, chronic lymphocytic leukemia, juvenile myelomonocytic leukemia, minimal residual disease, hairy cell leukemia, primary myelofibrosis, secondary myelofibrosis, chronic myeloid leukemia, myelodysplastic syndrome, myeloproliferative disease, or Waldestrom's macroglobulinemia.

In various embodiments, the cancer is pancreatic cancer, urological cancer, bladder cancer, colorectal cancer, colon cancer, breast cancer, prostate cancer, renal cancer, hepatocellular cancer, thyroid cancer, gall bladder cancer, lung cancer (e.g. non-small cell lung cancer, small-cell lung cancer), ovarian cancer, cervical cancer, gastric cancer, endometrial cancer, esophageal cancer, head and neck cancer, melanoma, neuroendocrine cancer, CNS cancer, brain tumors (e.g., glioma, anaplastic oligodendroglioma, adult glioblastoma multiforme, and adult anaplastic astrocytoma), bone cancer, soft tissue sarcoma, retinoblastomas, neuroblastomas, peritoneal effusions, malignant pleural effusions, mesotheliomas, Wilms tumors, trophoblastic neoplasms, hemangiopericytomas, Kaposi's sarcomas, myxoid carcinoma, round cell carcinoma, squamous cell carcinomas, esophageal squamous cell carcinomas, oral carcinomas, cancers of the adrenal cortex, or ACTH-producing tumors.

DLBCL is a diverse group of genetically heterogeneous tumors and represents the most common type of non-Hodgkin's lymphomas in adults. Transcriptional profiling studies have defined two subtypes of DLBCL: the activated B cell, or ABC, subtype signature is characterized by a gene expression signature similar to that of activated normal B-cell; the germinal center B cell, or GCB subtype gene expression signature resembles that of normal germinal center derived B-cell.

In one embodiment, the methods described herein are provided for treating lymphoma. In one embodiment, the methods described herein are provided for treating DLBCL. In one embodiment, the methods described herein are provided for treating FL. In one embodiment, the methods described herein are provided for treating non-Hodgkin's lymphoma or mantle cell lymphoma (MCL).

In one embodiment, the cancers treated by the disclosed methods have overexpression of Myc or Bcl-2. In one embodiment, the cancers treated by the disclosed methods have a translocation of Myc or Bcl-2. In one embodiment, the patient has DLBCL or MCL with overexpression or translation of Myc, Bcl-2 or the combination thereof.

Subject

The human in need thereof may be an individual who has or is suspected of having a cancer. In one embodiment, the human is at risk of developing a cancer (e.g., a human who is genetically or otherwise predisposed to developing a cancer) and has or has not been diagnosed with the cancer. As used herein, an “at risk” subject is a subject who is at risk of developing cancer (e.g., a hematologic malignancy). The subject may or may not have detectable disease, and may or may not have displayed detectable disease prior to the treatment methods described herein. An at risk subject may have one or more so-called risk factors, which are measurable parameters that correlate with development of cancer, such as described herein. A subject having one or more of these risk factors has a higher probability of developing cancer than an individual without these risk factor(s).

These risk factors may include, for example, age, sex, race, diet, history of previous disease, presence of precursor disease, genetic (e.g., hereditary) considerations, and environmental exposure. In one embodiment, a human at risk for cancer includes, for example, a human whose relatives have experienced this disease, and those whose risk is determined by analysis of genetic or biochemical markers. Prior history of having cancer may also be a risk factor for instances of cancer recurrence.

In one embodiment, provided herein is a method for treating a human who exhibits one or more symptoms associated with cancer (e.g., a hematologic malignancy). In one embodiment, the human is at an early stage of cancer. In other embodiments, the human is at an advanced stage of cancer.

In one embodiment, provided herein is a method for treating a human who is undergoing one or more standard therapies for treating cancer (e.g., a hematologic malignancy), such as chemotherapy, radiotherapy, immunotherapy, or surgery. Thus, in some foregoing embodiments, the combination of a Bcl-2 inhibitor and a BET inhibitor, as disclosed herein, may be administered before, during, or after administration of chemotherapy, radiotherapy, immunotherapy, or surgery.

In one embodiment, provided herein is a method for treating a human who is “refractory” to a cancer treatment or who is in “relapse” after treatment for cancer (e.g., a hematologic malignancy). A subject “refractory” to an anti-cancer therapy means they do not respond to the particular treatment, also referred to as resistant. The cancer may be resistant to treatment from the beginning of treatment, or may become resistant during the course of treatment, for example after the treatment has shown some effect on the cancer, but not enough to be considered a remission or partial remission. A subject in “relapse” means that the cancer has returned or the signs and symptoms of cancer have returned after a period of improvement, e.g. after a treatment has shown effective reduction in the cancer, such as after a subject is in remission or partial remission.

In one embodiment, the human is (i) refractory to at least one anti-cancer therapy, or (ii) in relapse after treatment with at least one anti-cancer therapy, or both (i) and (ii). In some of embodiments, the human is refractory to at least two, at least three, or at least four anti-cancer therapies (including, for example, standard or experimental chemotherapies).

In one embodiment, provided is a method for sensitizing a human who is (i) refractory to at least one chemotherapy treatment, or (ii) in relapse after treatment with chemotherapy, or both (i) and (ii), wherein the method comprises administering a Bcl-2 inhibitor in combination with a BET inhibitor, as disclosed herein, to the human. A human who is sensitized is a human who is responsive to the treatment involving administration of a Bcl-2 inhibitor in combination with a BET inhibitor, as disclosed herein, or who has not developed resistance to such treatment.

In one embodiment, provided herein is a methods for treating a human for a cancer, with comorbidity, wherein the treatment is also effective in treating the comorbidity. A “comorbidity” to cancer is a disease that occurs at the same time as the cancer.

Kits

Compositions (including, for example, formulations and unit dosages) comprising a Bcl-2 inhibitor, as disclosed herein, and compositions comprising a BET inhibitor, as disclosed herein, can be prepared and placed in an appropriate container, and labeled for treatment of an indicated condition. Accordingly, provided is also an article of manufacture, such as a container comprising a unit dosage form of a Bcl-2 inhibitor and a unit dosage form of a BET inhibitor, as disclosed herein, and a label containing instructions for use of the compounds. In one embodiment, the article of manufacture is a container comprising (i) a unit dosage form of a Bcl-2 inhibitor, as disclosed herein, and one or more pharmaceutically acceptable carriers, adjuvants or excipients; and (ii) a unit dosage form of a BET inhibitor, as disclosed herein, and one or more pharmaceutically acceptable carriers, adjuvants or excipients. In one embodiment, the unit dosage form for both the Bcl-2 inhibitor and the BET inhibitor is a tablet or a capsule.

Kits also are contemplated. For example, a kit can comprise unit dosage forms of a Bcl-2 inhibitor, as disclosed herein, and unit dosage forms of a BET inhibitor, as disclosed herein, and a package insert containing instructions for use of the kit in treatment of a medical condition. In one embodiment, the kit comprises (i) a unit dosage form of the Bcl-2 inhibitor, as disclosed herein, and one or more pharmaceutically acceptable carriers, adjuvants or excipients; and (ii) a unit dosage form of a BET inhibitor, as disclosed herein, and one or more pharmaceutically acceptable carriers, adjuvants or excipients. In one embodiment, the unit dosage form for both the Bcl-2 inhibitor and the BET inhibitor is a tablet or capsule.

The instructions for use in the kit may be for treating a cancer, including, for example, a hematologic malignancy, as further described herein.

In one embodiment, this disclosure provides a kit comprising (i) a pharmaceutical composition comprising a Bcl-2 inhibitor; (ii) a pharmaceutical composition comprising a BET inhibitor as disclosed herein; and (iii) instructions for use of the Bcl-2 inhibitor and the BET inhibitor in treating cancer.

### Additional Chemotherapeutic Agents

In one embodiment, the compositions described herein or the methods described herein can further include a third chemotherapeutic agent. As used herein, the term “chemotherapeutic agent” or “chemotherapeutic” (or “chemotherapy” in the case of treatment with a chemotherapeutic agent) is meant to encompass any non-proteinaceous (i.e., non-peptidic) chemical compound useful in the treatment of cancer.

Chemotherapeutic agents may be categorized by their mechanism of action into, for example, the following groups:
anti-metabolites/anti-cancer agents such as pyrimidine analogs floxuridine, capecitabine, and cytarabine;
purine analogs, folate antagonists, and related inhibitors;
antiproliferative/antimitotic agents including natural products such as vinca alkaloid (vinblastine, vincristine) and microtubule such as taxane (paclitaxel, docetaxel), vinblastin, nocodazole, epothilones, vinorelbine (NAVELBINE®), and epipodophyllotoxins (etoposide, teniposide);
DNA damaging agents such as actinomycin, amsacrine, busulfan, carboplatin, chlorambucil, cisplatin, cyclophosphamide (CYTOXAN®), dactinomycin, daunorubicin, doxorubicin, epirubicin, iphosphamide, melphalan, merchlorethamine, mitomycin, mitoxantrone, nitrosourea, procarbazine, taxol, taxotere, teniposide, etoposide, and triethylenethiophosphoramide;
antibiotics such as dactinomycin, daunorubicin, doxorubicin, idarubicin, anthracyclines, mitoxantrone, bleomycins, plicamycin (mithramycin), and mitomycin;
enzymes such as L-asparaginase which systemically metabolizes L-asparagine and deprives cells which do not have the capacity to synthesize their own asparagine;
antiplatelet agents;
antiproliferative/antimitotic alkylating agents such as nitrogen mustards cyclophosphamide and analogs (melphalan, chlorambucil, hexamethylmelamine, and thiotepa), alkyl nitrosoureas (carmustine) and analogs, streptozocin, and triazenes (dacarbazine);
antiproliferative/antimitotic antimetabolites such as folic acid analogs (methotrexate);
platinum coordination complexes (cisplatin, oxiloplatinim, and carboplatin), procarbazine, hydroxyurea, mitotane, and aminoglutethimide;
hormones, hormone analogs (estrogen, tamoxifen, goserelin, bicalutamide, and nilutamide), and aromatase inhibitors (letrozole and anastrozole);
anticoagulants such as heparin, synthetic heparin salts, and other inhibitors of thrombin;
fibrinolytic agents such as tissue plasminogen activator, streptokinase, urokinase, aspirin, dipyridamole, ticlopidine, and clopidogrel;
antimigratory agents;
antisecretory agents (breveldin);
immunosuppressives tacrolimus, sirolimus, azathioprine, and mycophenolate;
compounds (TNP-470, genistein) and growth factor inhibitors (vascular endothelial growth factor inhibitors and fibroblast growth factor inhibitors);
angiotensin receptor blockers, nitric oxide donors;
anti-sense oligonucleotides;
antibodies such as trastuzumab and rituximab;
cell cycle inhibitors and differentiation inducers such as tretinoin;
inhibitors, topoisomerase inhibitors (doxorubicin, daunorubicin, dactinomycin, eniposide, epirubicin, etoposide, idarubicin, irinotecan, mitoxantrone, topotecan, and irinotecan), and corticosteroids (cortisone, dexamethasone, hydrocortisone, methylprednisolone, prednisone, and prednisolone);
growth factor signal transduction kinase inhibitors;
dysfunction inducers;
toxins such as Cholera toxin, ricin, Pseudomonas exotoxin, Bordetella pertussis adenylate cyclase toxin, diphtheria toxin, and caspase activators;
and chromatin.

Further examples of chemotherapeutic agents include:
alkylating agents such as thiotepa and cyclophosphamide (CYTOXAN®);
alkyl sulfonates such as busulfan, improsulfan, and piposulfan;
aziridines such as benzodopa, carboquone, meturedopa, and uredopa;
emylerumines and memylamelamines including alfretamine, triemylenemelamine, triethylenephosphoramide, triethylenethi ophosphoramide, and trimemylolomelamine;
acetogenins, especially bullatacin and bullatacinone;
a camptothecin, including synthetic analog topotecan;
bryostatin;
callystatin;
CC-1065, including its adozelesin, carzelesin, and bizelesin synthetic analogs;
cryptophycins, particularly cryptophycin 1 and cryptophycin 8;
dolastatin;
duocarmycin, including the synthetic analogs KW-2189 and CBI-TMI;
eleutherobin;
pancratistatin;
a sarcodictyin;
spongistatin;
nitrogen mustards such as chlorambucil, chlornaphazine, cyclophosphamide, estramustine, ifosfamide, mechlorethamine, mechlorethamine oxide hydrochloride, melphalan, novembichin, phenesterine, prednimustine, trofosfamide, and uracil mustard;
nitrosoureas such as carmustine, chlorozotocin, foremustine, lomustine, nimustine, and ranimustine;
antibiotics such as the enediyne antibiotics (e.g., calicheamicin, especially calicheamicin gammaII and calicheamicin phiI1), dynemicin including dynemicin A, bisphosphonates such as clodronate, an esperamicin, neocarzinostatin chromophore and related chromoprotein enediyne antibiotic chromomophores, aclacinomycins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin, and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, and zorubicin;
anti-metabolites such as methotrexate and 5-fluorouracil (5-FU);
folic acid analogs such as demopterin, methotrexate, pteropterin, and trimetrexate;
purine analogs such as fludarabine, 6-mercaptopurine, thiamiprine, and thioguanine;
pyrimidine analogs such as ancitabine, azacitidine, 6-azauridine, carmofur, cytarabine, dideoxyuridine, doxifluridine, enocitabine, and floxuridine;
androgens such as calusterone, dromostanolone propionate, epitiostanol, mepitiostane, and testolactone;
anti-adrenals such as aminoglutethimide, mitotane, and trilostane;
folic acid replinishers such as frolinic acid;
trichothecenes, especially T-2 toxin, verracurin A, roridin A, and anguidine;
taxoids such as paclitaxel (TAXOL®) and docetaxel (TAXOTERE®);
platinum analogs such as cisplatin and carboplatin;
aceglatone; aldophosphamide glycoside; aminolevulinic acid; eniluracil; amsacrine; hestrabucil; bisantrene; edatraxate; defofamine; demecolcine; diaziquone; elformthine; elliptinium acetate; an epothilone; etoglucid; gallium nitrate; hydroxyurea; lentinan; leucovorin; lonidamine; maytansinoids such as maytansine and ansamitocins; mitoguazone; mitoxantrone; mopidamol; nitracrine; pentostatin; phenamet; pirarubicin; losoxantrone; fluoropyrimidine; folinic acid; podophyllinic acid; 2-ethylhydrazide; procarbazine; polysaccharide-K (PSK); razoxane; rhizoxin; sizofiran; spirogermanium; tenuazonic acid; triaziquone; 2,2′,2″-tricUorotriemylamine; urethane; vindesine; dacarbazine; mannomustine; mitobronitol; mitolactol; pipobroman; gacytosine; arabinoside (“Ara-C”); cyclophosphamide; thiopeta; chlorambucil; gemcitabine (GEMZAR®); 6-thioguanine; mercaptopurine; methotrexate; vinblastine; platinum; etoposide (VP-16); ifosfamide; mitroxantrone; vancristine; vinorelbine (NAVELBINE®); novantrone; teniposide; edatrexate; daunomycin; aminopterin; xeoloda; ibandronate; CPT-11; topoisomerase inhibitor RFS 2000; difluoromethylornithine (DFMO); retinoids such as retinoic acid; capecitabine; FOLFIRI (fluorouracil, leucovorin, and irinotecan);
and pharmaceutically acceptable salts, acids, or derivatives of any of the above.

Anti-Hormonal Agents

Also included in the definition of“chemotherapeutic agent” are anti-hormonal agents such as anti-estrogens and selective estrogen receptor modulators (SERMs), inhibitors of the enzyme aromatase, anti-androgens, and pharmaceutically acceptable salts, acids or derivatives of any of the above that act to regulate or inhibit hormone action on tumors.

Examples of anti-estrogens and SERMs include, for example, tamoxifen (including NOLVADEX™), raloxifene, droloxifene, 4-hydroxytamoxifen, trioxifene, keoxifene, LY117018, onapristone, and toremifene (FARESTON®).

Inhibitors of the enzyme aromatase regulate estrogen production in the adrenal glands. Examples include 4(5)-imidazoles, aminoglutethimide, megestrol acetate (MEGACE®), exemestane, formestane, fadrozole, vorozole (RIVISOR®), letrozole (FEMARA®), and anastrozole (ARIMIDEX®).

Examples of anti-androgens include flutamide, nilutamide, bicalutamide, leuprohde, and goserelin.

Anti-Angiogenic Agents

Anti-angiogenic agents include, but are not limited to, retinoid acid and derivatives thereof, 2-methoxyestradiol, ANGIOSTATIN®, ENDOSTATIN®, suramin, squalamine, tissue inhibitor of metalloproteinase-1, tissue inhibitor of metalloproteinase-2, plasminogen activator inhibitor-1, plasminogen activator inbibitor-2, cartilage-derived inhibitor, paclitaxel (nab-paclitaxel), platelet factor 4, protamine sulphate (clupeine), sulphated chitin derivatives (prepared from queen crab shells), sulphated polysaccharide peptidoglycan complex (sp-pg), staurosporine, modulators of matrix metabolism including proline analogs ((1-azetidine-2-carboxylic acid (LACA)), cishydroxyproline, d,I-3,4-dehydroproline, thiaproline, α,α′-dipyridyl, beta-aminopropionitrile fumarate, 4-propyl-5-(4-pyridinyl)-2(3h)-oxazolone, methotrexate, mitoxantrone, heparin, interferons, 2 macroglobulin-serum, chicken inhibitor of metalloproteinase-3 (ChIMP-3), chymostatin, beta-cyclodextrin tetradecasulfate, eponemycin, fumagillin, gold sodium thiomalate, d-penicillamine, beta-1-anticollagenase-serum, alpha-2-antiplasmin, bisantrene, lobenzarit disodium, n-2-carboxyphenyl-4-chloroanthronilic acid disodium or “CCA”, thalidomide, angiostatic steroid, carboxy aminoimidazole, and metalloproteinase inhibitors such as BB-94. Other anti-angiogenesis agents include antibodies, preferably monoclonal antibodies against these angiogenic growth factors: beta-FGF, alpha-FGF, FGF-5, VEGF isoforms, VEGF-C, HGF/SF, and Ang-1/Ang-2.

Anti-Fibrotic Agents

Anti-fibrotic agents include, but are not limited to, the compounds such as beta-aminoproprionitrile (BAPN), as well as the compounds disclosed in U.S. Pat. No. 4,965,288 (Palfreyman, et al.) relating to inhibitors of lysyl oxidase and their use in the treatment of diseases and conditions associated with the abnormal deposition of collagen and U.S. Pat. No. 4,997,854 (Kagan et al.) relating to compounds which inhibit LOX for the treatment of various pathological fibrotic states, which are herein incorporated by reference. Further exemplary inhibitors are described in U.S. Pat. No. 4,943,593 (Palfreyman et al.) relating to compounds such as 2-isobutyl-3-fluoro-, chloro-, or bromo-allylamine, U.S. Pat. No. 5,021,456 (Palfreyman et al.), U.S. Pat. No. 5,059,714 (Palfreyman et al.), U.S. Pat. No. 5,120,764 (Mccarthy et al.), U.S. Pat. No. 5,182,297 (Palfreyman et al.), U.S. Pat. No. 5,252,608 (Palfreyman et al.) relating to 2-(1-naphthyloxymemyl)-3-fluoroallylamine, and U.S. Pub. No.: 2004/0248871 (Farjanel et al.), which are herein incorporated by reference.

Exemplary anti-fibrotic agents also include the primary amines reacting with the carbonyl group of the active site of the lysyl oxidases, and more particularly those which produce, after binding with the carbonyl, a product stabilized by resonance, such as the following primary amines: emylenemamine, hydrazine, phenylhydrazine, and their derivatives; semicarbazide and urea derivatives; aminonitriles such as BAPN or 2-nitroethylamine; unsaturated or saturated haloamines such as 2-bromo-ethylamine, 2-chloroethylamine, 2-trifluoroethylamine, 3-bromopropylamine, and p-halobenzylamines; and selenohomocysteine lactone.

Other anti-fibrotic agents are copper chelating agents penetrating or not penetrating the cells. Exemplary compounds include indirect inhibitors which block the aldehyde derivatives originating from the oxidative deamination of the lysyl and hydroxylysyl residues by the lysyl oxidases. Examples include the thiolamines, particularly D-penicillamine, and its analogs such as 2-amino-5-mercapto-5-methylhexanoic acid, D-2-amino-3-methyl-3-((2-acetamidoethyl)dithio)butanoic acid, p-2-amino-3-methyl-3-((2-aminoethyl)dithio)butanoic acid, sodium-4-((p-1-dimethyl-2-amino-2-carboxyethyl)dithio)butane sulphurate, 2-acetamidoethyl-2-acetamidoethanethiol sulphanate, and sodium-4-mercaptobutanesulphinate trihydrate.

Immunotherapeutic Agents

The immunotherapeutic agents include and are not limited to therapeutic antibodies suitable for treating patients. Some examples of therapeutic antibodies include simtuzumab, abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farletuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, narnatumab, naptumomab, necitumumab, nimotuzumab, nofetumomab, ocaratuzumab, ofatumumab, olaratumab, onartuzumab, oportuzumab, oregovomab, panitumumab, parsatuzumab, patritumab, pemtumomab, pertuzumab, pintumomab, pritumumab, racotumomab, radretumab, rilotumumab, rituximab, robatumumab, satumomab, sibrotuzumab, siltuximab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49, and 3F8. Rituximab can be used for treating indolent B-cell cancers, including marginal-zone lymphoma, WM, CLL and small lymphocytic lymphoma. A combination of Rituximab and chemotherapy agents is especially effective.

The exemplified therapeutic antibodies may be further labeled or combined with a radioisotope particle such as indium-111, yttrium-90, or iodine-131.

In a certain embodiment, the additional therapeutic agent is a nitrogen mustard alkylating agent. Nonlimiting examples of nitrogen mustard alkylating agents include chlorambucil.

Lymphoma or Leukemia Combination Therapy

Some chemotherapy agents are suitable for treating lymphoma or leukemia. These agents include aldesleukin, alvocidib, antineoplaston AS2-1, antineoplaston A10, anti-thymocyte globulin, amifostine trihydrate, aminocamptothecin, arsenic trioxide, beta alethine, Bcl-2 family protein inhibitor navitoclax (ABT-263), venetoclax (ABT-199), ABT-737, BMS-345541, bortezomib (VELCADE®), bryostatin 1, busulfan, carboplatin, campath-1H, CC-5103, carmustine, caspofungin acetate, clofarabine, cisplatin, cladribine, chlorambucil, curcumin, cyclosporine, cyclophosphamide, cytarabine, denileukin diftitox, dexamethasone, DT-PACE (dexamethasone, thalidomide, cisplatin, doxorubicin, cyclophosphamide, and etoposide), docetaxel, dolastatin 10, doxorubicin, doxorubicin hydrochloride, enzastaurin, epoetin alfa, etoposide, everolimus (RAD001), fenretinide, filgrastim, melphalan, mesna, flavopiridol, fludarabine, geldanamycin (17-AAG), ifosfamide, irinotecan hydrochloride, ixabepilone, lenalidomide (REVLIMID®, CC-5013), lymphokine-activated killer cells, melphalan, methotrexate, mitoxantrone hydrochloride, motexafin gadolinium, mycophenolate mofetil, nelarabine, oblimersen, obatoclax (GX15-070), oblimersen, octreotide acetate, omega-3 fatty acids, oxaliplatin, paclitaxel, PD0332991, PEGylated liposomal doxorubicin hydrochloride, pegfilgrastim, pentostatin, perifosine, prednisolone, prednisone, R-roscovitine (seliciclib, CYC202), recombinant interferon alfa, recombinant interleukin-12, recombinant interleukin-11, recombinant flt3 ligand, recombinant human thrombopoietin, rituximab, sargramostim, sildenafil citrate, simvastatin, sirolimus, styryl sulphones, tacrolimus, tanespimycin, temsirolimus (CCI-779), thalidomide, therapeutic allogeneic lymphocytes, thiotepa, tipifarnib, bortezomib (VELCADE®, PS-341), vincristine, vincristine sulfate, vinorelbine ditartrate, SAHA (suberanilohydroxamic acid, or suberoyl, anilide, and hydroxamic acid), FR (fludarabine and rituximab), CHOP (cyclophosphamide, doxorubicin, vincristine, and prednisone), CVP (cyclophosphamide, vincristine, and prednisone), FCM (fludarabine, cyclophosphamide, and mitoxantrone), FCR (fludarabine, cyclophosphamide, and rituximab), hyperCVAD (hyperfractionated cyclophosphamide, vincristine, doxorubicin, dexamethasone, methotrexate, and cytarabine), ICE (iphosphamide, carboplatin, and etoposide), MCP (mitoxantrone, chlorambucil, and prednisolone), R-CHOP (rituximab and CHOP), R-CVP (rituximab and CVP), R-FCM (rituximab and FCM), R-ICE (rituximab and ICE), and R-MCP (rituximab and MCP).

One modified approach is radioimmunotherapy, wherein a monoclonal antibody is combined with a radioisotope particle, such as indium-111, yttrium-90, and iodine-131. Examples of combination therapies include, but are not limited to, iodine-131 tositumomab (BEXXAR®), yttrium-90 ibritumomab tiuxetan (ZEVALIN®), and BEXXAR® with CHOP.

The abovementioned therapies can be supplemented or combined with stem cell transplantation or treatment. Therapeutic procedures include peripheral blood stem cell transplantation, autologous hematopoietic stem cell transplantation, autologous bone marrow transplantation, antibody therapy, biological therapy, enzyme inhibitor therapy, total body irradiation, infusion of stem cells, bone marrow ablation with stem cell support, in vitro-treated peripheral blood stem cell transplantation, umbilical cord blood transplantation, immunoenzyme technique, low-LET cobalt-60 gamma ray therapy, bleomycin, conventional surgery, radiation therapy, and nonmyeloablative allogeneic hematopoietic stem cell transplantation.

Non-Hodgkin's Lymphomas Combination Therapy

Treatment of non-Hodgkin's lymphomas (NHL), especially those of B cell origin, includes using monoclonal antibodies, standard chemotherapy approaches (e.g., CHOP, CVP, FCM, MCP, and the like), radioimmunotherapy, and combinations thereof, especially integration of an antibody therapy with chemotherapy.

Examples of unconjugated monoclonal antibodies for the treatment of NHL/B-cell cancers include rituximab, alemtuzumab, human or humanized anti-CD20 antibodies, lumiliximab, anti-TNF-related apoptosis-inducing ligand (anti-TRAIL), bevacizumab, galiximab, epratuzumab, SGN-40, and anti-CD74.

Examples of experimental antibody agents used in treatment of NHL/B-cell cancers include ofatumumab, ha20, PRO131921, alemtuzumab, galiximab, SGN-40, CHIR-12.12, epratuzumab, lumiliximab, apolizumab, milatuzumab, and bevacizumab.

Examples of standard regimens of chemotherapy for NHL/B-cell cancers include CHOP, FCM, CVP, MCP, R—CHOP, R—FCM, R-CVP, and R-MCP.

Examples of radioimmunotherapy for NHL/B-cell cancers include yttrium-90 ibritumomab tiuxetan (ZEVALIN®) and iodine-131 tositumomab (BEXXAR®).

Mantle Cell Lymphoma Combination Therapy

Therapeutic treatments for mantle cell lymphoma (MCL) include combination chemotherapies such as CHOP, hyperCVAD, and FCM. These regimens can also be supplemented with the monoclonal antibody rituximab to form combination therapies R-CHOP, hyperCVAD-R, and R-FCM. Any of the abovementioned therapies may be combined with stem cell transplantation or ICE in order to treat MCL.

An alternative approach to treating MCL is immunotherapy. One immunotherapy uses monoclonal antibodies like rituximab. Another uses cancer vaccines, such as GTOP-99, which are based on the genetic makeup of an individual patient's tumor.

A modified approach to treat MCL is radioimmunotherapy, wherein a monoclonal antibody is combined with a radioisotope particle, such as iodine-131 tositumomab (BEXXAR®) and yttrium-90 ibritumomab tiuxetan (ZEVALIN®). In another example, BEXXAR® is used in sequential treatment with CHOP.

Other approaches to treating MCL include autologous stem cell transplantation coupled with high-dose chemotherapy, administering proteasome inhibitors such as bortezomib (VELCADE® or PS-341), or administering antiangiogenesis agents such as thalidomide, especially in combination with rituximab.

Another treatment approach is administering drugs that lead to the degradation of Bcl-2 protein and increase cancer cell sensitivity to chemotherapy, such as oblimersen, in combination with other chemotherapeutic agents.

A further treatment approach includes administering mTOR inhibitors, which can lead to inhibition of cell growth and even cell death. Non-limiting examples are temsirolimus (TORISEL®, CCI-779) and temsirolimus in combination with RITUXAN®, VELCADE®, or other chemotherapeutic agents.

Other recent therapies for MCL have been disclosed. Such examples include flavopiridol, PD0332991, R-roscovitine (selicicilib, CYC202), styryl sulphones, obatoclax (GX15-070), TRAIL, Anti-TRAIL death receptors DR4 and DR5 antibodies, temsirolimus (TORISEL®, CCI-779), everolimus (RAD001), BMS-345541, curcumin, SAHA, thalidomide, lenalidomide (REVLIMID®, CC-5013), and geldanamycin (17-AAG).

Diffuse Large B-Cell Lymphoma Combination Therapy

Therapeutic agents used to treat diffuse large B-cell lymphoma (DLBCL) include cyclophosphamide, doxorubicin, vincristine, prednisone, anti-CD20 monoclonal antibodies, etoposide, bleomycin, many of the agents listed for WM, and any combination thereof, such as ICE and R-ICE.

Chronic Lymphocytic Leukemia Combination Therapy

Examples of therapeutic agents used to treat chronic lymphocytic leukemia (CLL) include chlorambucil, cyclophosphamide, fludarabine, pentostatin, cladribine, doxorubicin, vincristine, prednisone, prednisolone, alemtuzumab, many of the agents listed for WM, and combination chemotherapy and chemoimmunotherapy, including the following common combination regimens: CVP, R-CVP, ICE, R-ICE, FCR, and FR.

Waldenstrom's Macroglobulinemia Combination Therapy

Therapeutic agents used to treat Waldenstrom's Macroglobulinemia (WM) include perifosine, bortezomib (VELCADE®), rituximab, sildenafil citrate (VIAGRA®), CC-5103, thalidomide, epratuzumab (hLL2-anti-CD22 humanized antibody), simvastatin, enzastaurin, campath-1H, dexamethasone, DT-PACE, oblimersen, antineoplaston A10, antineoplaston AS2-1, alemtuzumab, beta alethine, cyclophosphamide, doxorubicin hydrochloride, prednisone, vincristine sulfate, fludarabine, filgrastim, melphalan, recombinant interferon alfa, carmustine, cisplatin, cyclophosphamide, cytarabine, etoposide, melphalan, dolastatin 10, indium-111 monoclonal antibody MN-14, yttrium-90 humanized epratuzumab, anti-thymocyte globulin, busulfan, cyclosporine, methotrexate, mycophenolate mofetil, therapeutic allogeneic lymphocytes, yttrium-90 ibritumomab tiuxetan, sirolimus, tacrolimus, carboplatin, thiotepa, paclitaxel, aldesleukin, docetaxel, ifosfamide, mesna, recombinant interleukin-11, recombinant interleukin-12, Bcl-2 family protein inhibitor navitoclax, denileukin diftitox, tanespimycin, everolimus, pegfilgrastim, vorinostat, alvocidib, recombinant flt3 ligand, recombinant human thrombopoietin, lymphokine-activated killer cells, amifostine trihydrate, aminocamptothecin, irinotecan hydrochloride, caspofungin acetate, clofarabine, epoetin alfa, nelarabine, pentostatin, sargramostim, vinorelbine ditartrate, WT-1 analog peptide vaccine, WT1 126-134 peptide vaccine, fenretinide, ixabepilone, oxaliplatin, monoclonal antibody CD19, monoclonal antibody CD20, omega-3 fatty acids, mitoxantrone hydrochloride, octreotide acetate, tositumomab, iodine-131 tositumomab, motexafin gadolinium, arsenic trioxide, tipifarnib, autologous human tumor-derived HSPPC-96, veltuzumab, bryostatin 1, PEGylated liposomal doxorubicin hydrochloride, and any combination thereof.

Examples of therapeutic procedures used to treat WM include peripheral blood stem cell transplantation, autologous hematopoietic stem cell transplantation, autologous bone marrow transplantation, antibody therapy, biological therapy, enzyme inhibitor therapy, total body irradiation, infusion of stem cells, bone marrow ablation with stem cell support, in vitro-treated peripheral blood stem cell transplantation, umbilical cord blood transplantation, immunoenzyme techniques, low-LET cobalt-60 gamma ray therapy, bleomycin, conventional surgery, radiation therapy, and nonmyeloablative allogeneic hematopoietic stem cell transplantation.

Myelofibrosis Combination Therapy

Myelofibrosis inhibiting agents include, but are not limited to, hedgehog inhibitors, histone deacetylase (HDAC) inhibitors, and tyrosine kinase inhibitors. A non-limiting example of hedgehog inhibitors is saridegib.

Examples of HDAC inhibitors include, but are not limited to, pracinostat and panobinostat. A non-limiting example of a tyrosine kinase inhibitor is lestaurtinib.

Kinase Inhibitors

In one embodiment, the compounds described herein may be used or combined with one or more additional therapeutic agents. The one or more therapeutic agents include, but are not limited to, an inhibitor of Abl, activated CDC kinase (ACK), adenosine A2B receptor (A2B), apoptosis signal-regulating kinase (ASK), Auroa kinase, Bruton's tyrosine kinase (BTK), BET-bromodomain (BRD) such as BRD4, c-Kit, c-Met, CDK-activating kinase (CAK), calmodulin-dependent protein kinase (CaMK), cyclin-dependent kinase (CDK), casein kinase (CK), discoidin domain receptor (DDR), epidermal growth factor receptors (EGFR), focal adhesion kinase (FAK), Flt-3, FYN, glycogen synthase kinase (GSK), HCK, histone deacetylase (HDAC), IKK such as IKKβε, isocitrate dehydrogenase (IDH) such as IDH1, Janus kinase (JAK), KDR, lymphocyte-specific protein tyrosine kinase (LCK), lysyl oxidase protein, lysyl oxidase-like protein (LOXL), LYN, matrix metalloprotease (MMP), MEK, mitogen-activated protein kinase (MAPK), NEK9, NPM-ALK, p38 kinase, platelet-derived growth factor (PDGF), phosphorylase kinase (PK), polo-like kinase (PLK), phosphatidylinositol 3-kinase (PI3K), protein kinase (PK) such as protein kinase A, B, and/or C, PYK, spleen tyrosine kinase (SYK), serine/threonine kinase TPL2, serine/threonine kinase STK, signal transduction and transcription (STAT), SRC, serine/threonine-protein kinase (TBK) such as TBK1, TIE, tyrosine kinase (TK), vascular endothelial growth factor receptor (VEGFR), YES, or any combination thereof.

Apoptosis Signal-Regulating Kinase (ASK) Inhibitors

ASK inhibitors include ASK 1 inhibitors. Examples of ASK 1 inhibitors include, but are not limited to, those described in WO 2011/008709 (Gilead Sciences, Inc.) and WO 2013/112741 (Gilead Sciences, Inc.).

Bruton's Tyrosine Kinase (BTK) Inhibitors

Examples of BTK inhibitors include, but are not limited to, ibrutinib, HM71224, ONO-4059, and CC-292.

Discoidin Domain Receptor (DDR) Inhibitors

DDR inhibitors include inhibitors of DDR1 and/or DDR2. Examples of DDR inhibitors include, but are not limited to, those disclosed in WO 2014/047624 (Gilead Sciences, Inc.), US 2009/0142345 (Takeda Pharmaceutical), US 2011/0287011 (Oncomed Pharmaceuticals), WO 2013/027802 (Chugai Pharmaceutical), and WO 2013/034933 (Imperial Innovations).

Histone Deacetylase (HDAC) Inhibitors

Examples of HDAC inhibitors include, but are not limited to, pracinostat and panobinostat.

Janus Kinase (JAK) Inhibitors

JAK inhibitors inhibit JAK1, JAK2, and/or JAK3. Examples of JAK inhibitors include, but are not limited to, filgotinib, ruxolitinib, fedratinib, tofacitinib, baricitinib, lestaurtinib, pacritinib, XL019, AZD1480, INCB039110, LY2784544, BMS911543, and NS018.

Lysyl Oxidase-Like Protein (LOXL) Inhibitors LOXL inhibitors include inhibitors of LOXL1, LOXL2, LOXL3, LOXL4, and/or LOXL5. Examples of LOXL inhibitors include, but are not limited to, the antibodies described in WO 2009/017833 (Arresto Biosciences).

Examples of LOXL2 inhibitors include, but are not limited to, the antibodies described in WO 2009/017833 (Arresto Biosciences), WO 2009/035791 (Arresto Biosciences), and WO 2011/097513 (Gilead Sciences, Inc.).

Matrix Metalloprotease (MMP) Inhibitors

MMP inhibitors include inhibitors of MMP1 through 10. Examples of MMP9 inhibitors include, but are not limited to, marimastat (BB-2516), cipemastat (Ro 32-3555), and those described in WO 2012/027721 (Gilead Sciences, Inc.).

Phosphatidylinositol 3-Kinase (PI3K) Inhibitors

PI3K inhibitors include inhibitors of PI3Kγ, PI3Kδ, PI3Kβ, PI3Kα, and/or pan-PI3K. Examples of PI3K inhibitors include, but are not limited to, wortmannin, BKM120, CH5132799, XL756, and GDC-0980.

Examples of PI3Kγ inhibitors include, but are not limited to, ZSTK474, AS252424, LY294002, and TG100115.

Examples of PI3Kδ inhibitors include, but are not limited to, Compound B, Compound C, Compound D, Compound E. PI3K II, TGR-1202, AMG-319, GSK2269557, X-339, X-414, RP5090, KAR4141, XL499, OXY111A, IPI-145, IPI-443, and the compounds described in WO 2005/113556 (ICOS), WO 2013/052699 (Gilead Calistoga), WO 2013/116562 (Gilead Calistoga), WO 2014/100765 (Gilead Calistoga), WO 2014/100767 (Gilead Calistoga), and WO 2014/201409 (Gilead Sciences, Inc.).

Examples of PI3Kβ inhibitors include, but are not limited to, GSK2636771, BAY 10824391, and TGX221.

Examples of PI3Kα inhibitors include, but are not limited to, buparlisib, BAY 80-6946, BYL719, PX-866, RG7604, MLN1117, WX-037, AEZA-129, and PA799.

Examples of pan-PI3K inhibitors include, but are not limited to, LY294002, BEZ235, XL147 (SAR245408), and GDC-0941.

Spleen Tyrosine Kinase (SYK) Inhibitors

Examples of SYK inhibitors include, but are not limited to, tamatinib (R406), fostamatinib (R788), PRT062607, BAY-61-3606, NVP-QAB 205 AA, R112, R343, and those described in U.S. Pat. No. 8,450,321 (Gilead Connecticut).

Tyrosine-Kinase Inhibitors (TKIs)

TKIs may target epidermal growth factor receptors (EGFRs) and receptors for fibroblast growth factor (FGF), platelet-derived growth factor (PDGF), and vascular endothelial growth factor (VEGF). Examples of TKIs that target EGFR include, but are not limited to, gefitinib and erlotinib. Sunitinib is a non-limiting example of a TKI that targets receptors for FGF, PDGF, and VEGF.

### Pharmaceutical Compositions and Modes of Administration

In one embodiment, this disclosure provides a composition comprising a Bcl-2 inhibitor and a BET inhibitor. In one embodiment, this disclosure provides a composition comprising Bcl-2 inhibitor, a BET inhibitor, such as a compound of Formula (I) or a pharmaceutically acceptable salt, complex, solvate, prodrug, stereoisomer, mixture of stereoisomers or hydrate thereof, and a pharmaceutically acceptable carrier.

In one embodiment, this disclosure provides a co-formulation comprising a Bcl-2 inhibitor, a BET inhibitor, and a pharmaceutically acceptable carrier. In one embodiment, this disclosure provides a co-formulation comprising a Bcl-2 inhibitor, a therapeutically effective amount of a BET inhibitor, such as a compound of Formula (I) or a pharmaceutically acceptable salt, complex, solvate, prodrug, stereoisomer, mixture of stereoisomers or hydrate thereof, and a pharmaceutically acceptable carrier.

The pharmaceutical compositions or co-formulations may be administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

In one embodiment, the compounds described herein may be administered orally. Oral administration may be via, for example, capsule or enteric coated tablets. In making the pharmaceutical compositions that include at least one compound of Formula (I), or a pharmaceutically acceptable salt, complex, solvate, prodrug, stereoisomer, mixture of stereoisomers or hydrate thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

For preparing solid compositions such as tablets, the principal active ingredient may be mixed with a pharmaceutical carrier or excipient to form a solid preformulation composition containing a homogeneous mixture of a compound of Formula (I) or a pharmaceutically acceptable salt, complex, solvate, prodrug, stereoisomer, mixture of stereoisomers or hydrate thereof and/or a Bcl-2 inhibitor. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds disclosed herein may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Some examples of suitable carriers or excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl and propylhydroxy-benzoates; sweetening agents; and flavoring agents.

The compositions can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Another formulation for use in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. Such patches may be constructed for continuous, pulsatile, or on demand delivery of pharmaceutical agents.

Dosing

The dosing regimen of a Bcl-2 inhibitor (e.g., venetoclax, ABT-737, or navitoclax), and a BET inhibitor disclosed herein (e.g. a compound of Formula (I) or a pharmaceutically acceptable salt, complex, solvate, prodrug, stereoisomer, a mixture of stereoisomers or a hydrate thereof) in the methods provided herein may vary depending upon the indication, route of administration, and severity of the condition. For instance, depending on the route of administration, a suitable dose can be calculated according to body weight, body surface area, or organ size. The final dosing regimen is determined by the attending physician in view of good medical practice, considering various factors that modify the action of drugs, e.g., the specific activity of the compound, the identity and severity of the disease state, the responsiveness of the subject, the age, condition, body weight, sex, and diet of the subject, and the severity of any infection. Additional factors that can be taken into account include time and frequency of administration, drug combinations, reaction sensitivities, and tolerance/response to therapy. Further refinement of the doses appropriate for treatment involving any of the formulations mentioned herein is done routinely by the skilled practitioner without undue experimentation, especially in light of the dosing information and assays disclosed, as well as the pharmacokinetic data observed in human clinical trials. Appropriate doses can be ascertained through use of established assays for determining concentration of the agent in a body fluid or other sample together with dose response data.

As indicated above, the dose and frequency of dosing may depend on pharmacokinetic and pharmacodynamic, as well as toxicity and therapeutic efficiency data. For example, pharmacokinetic and pharmacodynamic information about the Bcl-2 inhibiting compounds (e.g., venetoclax, ABT-737, or navitoclax) and the BET inhibitor disclosed herein (e.g. a compound of Formula (I) or a pharmaceutically acceptable salt, complex, solvate, prodrug, stereoisomer, a mixture of stereoisomers or a hydrate thereof) can be collected through preclinical in vitro and in vivo studies, later confirmed in humans during the course of clinical trials. Thus, for the Bcl-2 inhibitors and the BET inhibitors disclosed herein, a therapeutically effective dose can be estimated initially from biochemical and/or cell-based assays. The dosage can then be formulated in animal models to achieve a desirable circulating concentration range that modulates BET activity. As human studies are conducted further information will emerge regarding the appropriate dosage levels and duration of treatment for various diseases and conditions.

Toxicity and therapeutic efficacy of a Bcl-2 inhibitor or a BET inhibitor can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the “therapeutic index”, which typically is expressed as the ratio LD₅₀/ED₅₀. Compounds that exhibit large therapeutic indices, i.e., the toxic dose is substantially higher than the effective dose, are preferred. The data obtained from such cell culture assays and additional animal studies can be used in formulating a range of dosage for human use. The doses of such compounds lies preferably within a range of circulating concentrations that include the ED₅₀ with little or no toxicity.

In one embodiment, a therapeutically effective amount or a pharmaceutically effective amount refers to an amount that is sufficient to effect treatment, when administered to a subject (e.g., a human) in need of such treatment. In one embodiment, a therapeutically effective amount of a Bcl-2 inhibitor is an amount sufficient to modulate Bcl-2 expression and/or activity, and thereby treat a human suffering an indication, or to ameliorate or alleviate the existing symptoms of the indication. In one embodiment, a therapeutically effective amount of a compound of Formula (I) or a pharmaceutically acceptable salt, complex, solvate, prodrug, stereoisomer, a mixture of stereoisomers or a hydrate thereof, is an amount sufficient to modulate activity of bromodomain-containing proteins, and thereby treat a human suffering an indication, or to ameliorate or alleviate the existing symptoms of the indication.

The dose administered of any of the compounds disclosed herein may be administered once daily (QD), twice daily (BID), three times daily, four times daily, or more than four times daily using any suitable mode described herein (e.g., oral administration). In one embodiment, the dose of any of the compounds disclosed herein is administered once daily. In one embodiment, the dose of any of the compounds disclosed herein is administered twice daily.

Moreover, administration or treatment with the compounds disclosed herein may be continued for a number of days; for example, treatment may continue for at least 7 days, 14 days, or 28 days, for one cycle of treatment. Treatment cycles are well known, and are frequently alternated with resting periods of about 1 to 28 days, commonly about 7 days or about 14 days, between cycles. The treatment cycles, in other embodiments, may also be continuous.

In one embodiment, a Bcl-2 inhibitor is administered to a human at a dose between 1 mg and 1000 mg, between 1 mg and 500 mg, between 10 mg and 500 mg, between 10 mg and 100 mg, between 10 mg and 80 mg, or between 10 mg and 50 mg. In one embodiment, the therapeutically effective amount of the Bcl-2 inhibitor, is administered to a human at a dose of about 1 mg, 10 mg, about 20 mg, about 30 mg, about 40 mg, about 50 mg, about 60 mg, about 70 mg, about 80 mg, about 90 mg, about 100 mg, about 200 mg, about 300 mg, about 400 mg, about 500 mg, about 600 mg, about 700 mg, about 800 mg, about 900 mg, about 1000 mg. In one embodiment, the dose of the Bcl-2 inhibitor disclosed herein is administered once. In one embodiment, the dose of the Bcl-2 inhibitor disclosed herein is administered twice daily.

In one embodiment, exemplary doses of the BET inhibitor, such as a compound of Formula (I) or a pharmaceutically acceptable salt, complex, solvate, prodrug, stereoisomer, mixture of stereoisomers or hydrate thereof, for a human subject may be from about 1 mg to about 100 mg, from about 1 mg to about 80 mg from about 10 mg to about 80 mg, or from about 10 mg to about 50 mg. In one embodiment, about 1 mg, about 10 mg, about 20 mg, about 30 mg, about 40 mg, about 50 mg, about 60 mg, about 70 mg, about 80 mg, about 90 mg, or about 100 mg. In one embodiment, the dose of the BET inhibitor disclosed herein is administered once. In one embodiment, the dose of the BET inhibitor disclosed herein is administered twice daily.

The compositions may, in one embodiment, be formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The compounds are generally administered in a pharmaceutically effective amount. In one embodiment, for oral administration, each dosage unit contains from about 1 mg to about 100 mg of a compound described herein, for example, about 1 mg, about 10 mg, about 20 mg, about 30 mg, about 40 mg, about 50 mg, about 60 mg, about 70 mg, about 80 mg, about 90 mg, or about 100 mg. It will be understood, however, that the amount of the compound actually administered usually will be determined by a physician, in the light of the relevant circumstances, including the condition to be treated, the chosen route of administration, the actual compound administered and its relative activity, the age, weight, and response of the individual subject, and the severity of the subject's symptoms.

### EXAMPLES

The following examples are provided to further aid in understanding the embodiments disclosed in the application, and presuppose an understanding of conventional methods well known to those persons having ordinary skill in the art to which the examples pertain. The particular materials and conditions described hereunder are intended to exemplify particular aspects of embodiments disclosed herein and should not be construed to limit the reasonable scope thereof.

### Example 1. Methods for Testing Combinations of BCL2 Inhibitor and BET Inhibitor in Lymphoma Cell Lines

This example provides materials and methods for evaluating the in vitro activity of the combination of venetoclax, a Bcl-2 inhibitor, with Compound A and B, two BET inhibitors, in a panel lymphoma cell lines as shown in Examples 2-5. Compound A is a compound of formula:

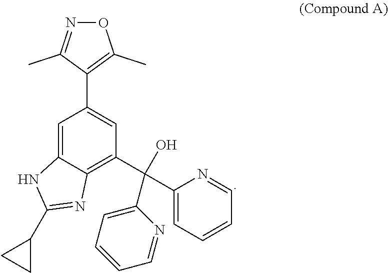 [SMILES: Cc1noc(C)c1-c1cc(C(O)(c2ccccn2)c2ccccn2)c2nc(C3CC3)[nH]c2c1]

The method of making Compound A is described in U.S. Patent Publication No.: 2014/0336190 A1 (Gilead Sciences, Inc.). Compound B is another BET inhibitor, which is a compound of formula:

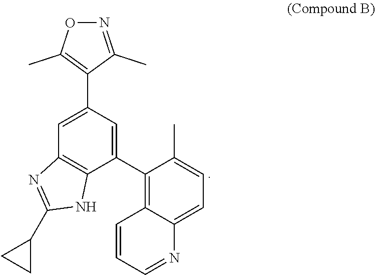 [SMILES: Cc1ccc2ncccc2c1-c1cc(-c2c(C)noc2C)cc2nc(C3CC3)[nH]c12]

Single Agent Compound A Cell Growth Assessment

Compound A was serially diluted by three-fold in 100% DMSO in a 96 well plate format in duplicate dose curves to achieve a dose range of 10 mM to 0.17 mM as a 1000× stock. Serially-diluted 1000× Compound A was then further diluted 1:100 in RPMI-1640 without additives to generate a 10× stock and finally diluted 1:10 into assay plate wells containing 100 μL of cells. The final concentration of DMSO was 0.1% in 111 μL of final volume in the assay wells and the dose range of Compound A was 10 μM to 0.5 nM. Cells were plated at 10,000 cells per well with the exceptions of cell lines SU-DHL-5 and OCI-Ly10, which were plated at 20,000 cells per well, SU-DHL-6 and SU-DHL-8 at 40,000 cells per well, and Toledo plated at 80,000 cells per well. Cells were plated in duplicate plates, two to four hours prior to compound addition. Following compound addition, assay plates were incubated at 37° C. in 5% CO₂ for four days in growth medium appropriate for each cell line. Cell lines SU-DHL-5, OCI-Ly10, SU-DHL-6, SU-DHL-8, and Toledo were incubated for three days. Cell viability was assessed using Cell Titer Glo (CTG, Promega, Madison, Wis.) following the manufacturer's protocol. Luminescence was measured on an EnSpire Multimode Plate Reader (Perkin Elmer, Waltham, Mass., Model 2300-001M).

A replicate assay plate was created at study initiation, processed immediately for cell viability by CTG, and these values represented 0% growth (day 0). Control wells on all assay plates included 0.1% DMSO (100% growth on day 3 or 4) and 1 μM doxorubicin (0% viability on day 3 or 4). The CTG signal for 1 μM doxorubicin on the last day was always smaller than the “day 0” signal for each cell line, with the respective ratio (Doxorubicin/CTG signal day 0) ranging from 0.003 to 0.577 across the 27-cell line panel, demonstrating that doxorubicin effectively killed each cell line at this concentration. Curve fittings were applied with a four parameter variable slope model and the following three calculations were determined using the curve fits. All values reported represent mean values of four to six determinations. The GI₅₀ was defined as the interpolated concentration that caused a 50% reduction in cell growth over the assay interval.

Annexin V Apoptosis Assay

Apoptosis induced by Compound A was measured by Annexin V staining as detected by flow cytometry. Compound A was diluted two-fold in duplicate to a starting concentration of 5 mM in 100% DMSO, and then serially diluted by three-fold in 100% DMSO in a 96 well plate format to achieve a 500× dose range of 5 mM to 0.76 μM. Serially-diluted Compound A was then further diluted 1:5 in sterile water to generate a 100× stock and finally diluted 1:100 into assay plate wells containing 200 μL of cells. The final concentration of DMSO was 0.2% in 200 μL in the assay wells and the dose range of Compound A was 10 μM to 1.5 nM. Cells were plated at 20,000-80,000 cells per well and assay plates were incubated after compound addition at 37° C. with 5% CO₂ for 3 days in growth medium appropriate for each cell line. Control wells on all assay plates included 0.2% DMSO (0% apoptosis on day 3), 1 μM Velcade®, and 1 μM staurosporine (apoptosis control on day 3). Velcade® and staurosporine were used as benchmarks to ensure apoptosis could be achieved in each cell line. The range of apoptosis was 60%-98% for staurosporine and 58% to 99% for Velcade® across the 27 cell lines.

After 72 to 96 hours, the cell compound mixture was transferred to a 96-well deep well conical bottom plate, centrifuged at 300×g, and cell pellets were re-suspended in DPBS. Plates were centrifuged at 300×g, supernatant was decanted, and cell pellets were re-suspended in 500 μl of pre-diluted LIVE/DEAD® Fixable Aqua Dead Cell Stain for 30 minutes at room temperature protected from light. Assay plates were centrifuged at 300×g, supernatant was decanted, and cell pellets were washed twice in 500 μL of DPBS containing 4% FBS. The washed pellets were re-suspended in 100 μL DPBS containing 4% FBS, 5 μL of Annexin V APC was added to each well, and assay plates were incubated for 30 minutes at room temperature, protected from light. Assay plates were washed twice with 500 μL of DPBS containing 4% FBS per well, and cell pellets were finally re-suspended in 250 μL of DPBS containing 4% FBS.

Flow cytometric sampling of 20,000 total events within a forward scatter and side-scatter gate (P1) per well were collected on a BD FACS Canto II instrument with DIVA software (San Jose, Calif.) using a high throughput screen auto-sampler for analysis of apoptosis. Within the P1 gate Annexin V⁺/LIVE/DEAD⁻ and Annexin V⁺/LIVE/DEAD⁺ cells were digitally monitored. Single- or double-labeled cells were gated, percentages of Annexin V-positive cells in each population were recorded and combined, and data were extracted to a flow cytometry standard file (fcs). Mean percentages of Annexin V-positive cells were determined for the positive control (Velcade® and staurosporine at 1 μM) and negative wells (0.2% DMSO) to ensure the assay performed as expected for each cell line on each experimental day.

EC₅₀ values for each compound were calculated based on log concentration of drug and percent Annexin V-positive cells using a four-parameter logistical fit equation in Prism 6.01 of the GraphPad software package. The percentage of apoptosis at EC₅₀ was interpolated from the curve fit for each cell line. Cell lines were determined to be sensitive to Compound A by induction of apoptosis if the number of Annexin V-positive cells at 1.1 μM Compound A was greater than 30% and there was a two-fold change over baseline apoptosis. For an experiment to be valid, the % baseline apoptosis for a given cell line was required to be <35%.

Western Blot Analysis

One FL and 14 DLBCL cell lines were grown logarithmically in appropriate growth conditions. Approximately 1.0×10⁶ cells were pelleted by centrifugation, washed once in 1×PBS and 1 mL of RIPA buffer (Cell Signaling Technologies, #9806) containing protease inhibitors (Pierce, #78430) was added to each pellet to generate lysates stored at −80° C. An equivalent of 40 μg of total protein from untreated DLBCL cell lysates was loaded on to 4-12% bis-tris gels and transferred on to nitrocellulose membrane using i-Blot transfer system. The blots were blocked with 5% milk and probed with primary Antibody at 4° C. overnight. The blots were washed 3 times with TBST buffer and probed with corresponding Licor fluorescent secondary antibody for one hour at RT. Subsequently, the blots were washed 3 times with TBST buffer and scanned in the Licor imaging system. The antibodies used for the westerns were Myc (CST; #5605), Bcl-2 (Invitrogen; #13-8800), Bcl-XL (CST; #2762), Mcl-1 (BD biosciences; #559027), BIM (CST; #2819) and ACTIN (sigma; #A5316).

Venetoclax EC₅₀ Analysis DLBCL cell lines were cultured according to ATCC or DSMZ recommended conditions to log phase growth and seeded in 96 well microtitre plates at 3000, 10,000, or 30,000 cells per well depending on previous optimization. Compounds were applied from 10 μM to 0.0015 μM at 9 three-fold dilutions and plates were incubated with cells for 96 hours at 37° C. 100% RH. Viability assessed by Promega Cell Titer Glo ATP assay and EC₅₀ results calculated in GraphPad Prism by four parameter curve fit of log [μM] vs raw luminescence units.

Synergy Screening

Cells were cultured in conditions as described in Table 2. Cells were seeded such that they were growing in log phase throughout the length of the 72 hour assay. Viability was determined by the ATPLite assay which quantifies ATP as an indicator of viable cells. DMSO-treated and Day 0 measurements were acquired and used to establish no cell growth inhibition (a value of 0) and the threshold of cytostatic behavior (a value of 100). Values above 100 denote cell death resulting in fewer cells than were present at the beginning of treatment and ranged from 100 (the same as the Day 0 value) to 200 (complete absence of viable cells).

TABLE 2

| Cell line | Subtype | Culture Conditions |
|---|---|---|
| CARNAVAL | double hit | RPMI with 20% FBS |
| GRANTA-519 | MCL | RPMI with 10% FBS |
| HBL-1 | ABC-DLBCL | IMDM with 10% FBS |
| Jeko-1 | MCL | RPMI with 20% FBS |
| JMP-1 | MCL | RPMI with 15% FBS |
| JVM-13 | MCL | RPMI with 10% FBS |
| JVM-2 | MCL | RPMI with 10% FBS |
| MAVER-1 | MCL | RPMI with 10% FBS |
| Mino | MCL | RPMI with 20% FBS |
| OCI-Ly10 | ABC-DLBCL | Iscoves with 20% human serum |
| OCI-Ly3 | ABC-DLBCL | Iscoves with 20% FBS |
| OCI-Ly4 | GCB-DLBCL | Iscoves with 20% FBS |
| PF-1 | MCL | RPMI with 15% FBS |
| PF-2 | MCL | RPMI with 15% FBS |
| Pfeiffer | GCB-DLBCL | RPMI with 10% FBS |
| REC-1 | MCL | RPMI with 10% FBS |
| Ri-1 | ABC-DLBCL | RPMI with 10% FBS |
| SU-DHL-10 | GCB-DLBCL | RPMI with 10% FBS |
| SU-DHL-2 | ABC-DLBCL | RPMI with 10% FBS |
| SU-DHL-4 | GCB-DLBCL | RPMI with 20% FBS |
| SU-DHI-5 | GCB-DLBCL | RPMI with 10% FBS |
| SU-DHL-6 | GCB-DLBCL | RPMI with 10% FBS |
| SU-DHL-8 | GCB-DLBCL | RPMI with 10% FBS |
| TMD8 | ABC-DLBCL | MEM alpha with 10% FBS |
| U-2932 | ABC-DLBCL | RPMI with 10% FBS |
| U-2973 | double hit | RPMI with 20% FBS |
| WSU-FSCCL | FL | RMPI 10% FBS |
| Z-138 | MCL | RPMI with 10% FBS |

To measure combination effects in excess of Loewe additivity, a scalar measure was devised to characterize the strength of synergistic interaction termed the synergy score. In one embodiment, the synergy score was calculated as:

Synergy Score=log fₓ log f_{y}Σmax(0,I_{data})(I_{data}−I_{Loewe})

The fractional inhibition for each component agent and combination point in the matrix is calculated relative to the median of all vehicle-treated control wells. The synergy score equation integrates the experimentally-observed activity volume at each point in the matrix in excess of a model surface numerically derived from the activity of the component agents using the Loewe model for additivity. Additional terms in the synergy score equation are used to normalize for various dilution factors used for individual agents and to allow for comparison of synergy scores across an entire experiment. The inclusion of positive inhibition gating or an I_{data} multiplier removes noise near the zero effect level, and biases results for synergistic interactions at that occur at high activity levels.

Potency shifting was evaluated using an isobologram, which demonstrates how much less drug is required in combination to achieve a desired effect level, when compared to the single agent doses needed to reach the effect. The isobologram was drawn by identifying the locus of concentrations that correspond to crossing the indicated inhibition level. This is done by finding the crossing point for each single agent concentration in a dose matrix across the concentrations of the other single agent. Practically, each vertical concentration C_{Y} is held fixed while a bisection algorithm is used to identify the horizontal concentration C_{X} in combination with that vertical dose that gives the chosen effect level in the response surface Z(C_{X},C_{Y}). These concentrations are then connected by linear interpolation to generate the isobologram display. For synergistic interactions, the isobologram contour fall below the additivity threshold and approaches the origin, and an antagonistic interaction would lie above the additivity threshold. The error bars represent the uncertainty arising from the individual data points used to generate the isobologram. The uncertainty for each crossing point is estimated from the response errors using bisection to find the concentrations where Z−σ_{Z}(C_{X},C_{Y}) and Z+σ_{Z}(C_{X},C_{Y}) cross I_{cut}, where σ_{Z} is the standard deviation of the residual error on the effect scale.

Extracting Cell Growth Inhibition with Error Data from Zalicus Chalice Tool for Bar Graph.

The Chalice software was used. Under the “Inhibition” view in the Chalice software, all growth inhibition values are plotted from 0 to 100 in a heatmap in a blue-green color scheme. Under the “select columns” tool bar to the left of the “filter” tab, “Dose Matrix Error” was selected, which added a column displaying the “Inhibition Error” for the dose matrix. The % inhibition were subtracted from 100 to give the % of living cells, and the error values for each pairwise combination were plotted as the SD within graphpad prism where the values for mean, n, and SD are entered manually. Therefor the visualized graph displays the mean+/−SEM.

Calculation of Clinically Relevant Doses

Clinically relevant exposures of venetoclax were based on the 400 mg dose in a Phase I NHL study, cell culture media (CCM)-adjusted Cₘᵢₙ=53 nM, Cₘₐₓ=536 nM). Clinically relevant doses of Compound A were calculated. These exposures are comparable to the protein and potency adjusted exposures of the OTX-015 MTD exposures in a Phase I NHL trial (Compound A RFD, CCM adjusted Cₘₐₓ=90 nM, Cₐᵥₑ=40 nM, Cₘᵢₙ=10 nM). Protein shift calculations are shown in Table 3.

TABLE 3
ABT-199 (MW 868.4)
based on the 400 mg dose
http://meetinglibrary.asco.org/content/92930?media=vm&poster=1
CCM/buffer 0.44%
HP/buffer 0.10%

| | Cmin | Cmax |
|---|---|---|
| exposures (ug/mL) | 0.2 | 2 |
| exposures (nM) | 230 | 2303 |
| CCM protein adjusted (nM) | 53.56 | 536.32 |

Compound A (MW = 437.5)
based on 4 mg, subject #2
CCM/buffer 67%
HP/buffer 11.7

| | Cmin | Cmax |
|---|---|---|
| exposures (ug/mL) | 0.106 | 0.279 |
| exposures (nM) | 242 | 638 |
| CCM protein adjusted CCM (nM) | 42.26 | 111.41 |

### Example 2. Cell Growth Inhibition and Induction of Apoptosis in Response to Compound A

The activity of Compound A to induce apoptosis or inhibit cell growth was assessed across a panel of DLBCL cell lines. DLBCL cell lines were subjected to a ten point dose response of Compound A (range 0.5 nM-10,000 nM, 3× dilution series) and assessed for cell growth inhibition by CTG or apoptosis by AnnexinV staining after three or four days. Of the twenty-one DLBCL and FL cell lines, a total of 11 cell lines underwent apoptosis in response to single agent Compound A and 10 did not (% apoptosis <30% by 1 μM). Eight were >30% Annexin V positive after treatment with 1 μM with EC₅₀ s<113 nM (approximately the protein adjusted Cₘₐₓ concentration of Compound A), and three with an EC₅₀ above 113 nM (apoptosis EC₅₀s equal to 274 nM, 881 nM, and 2.5 μM).

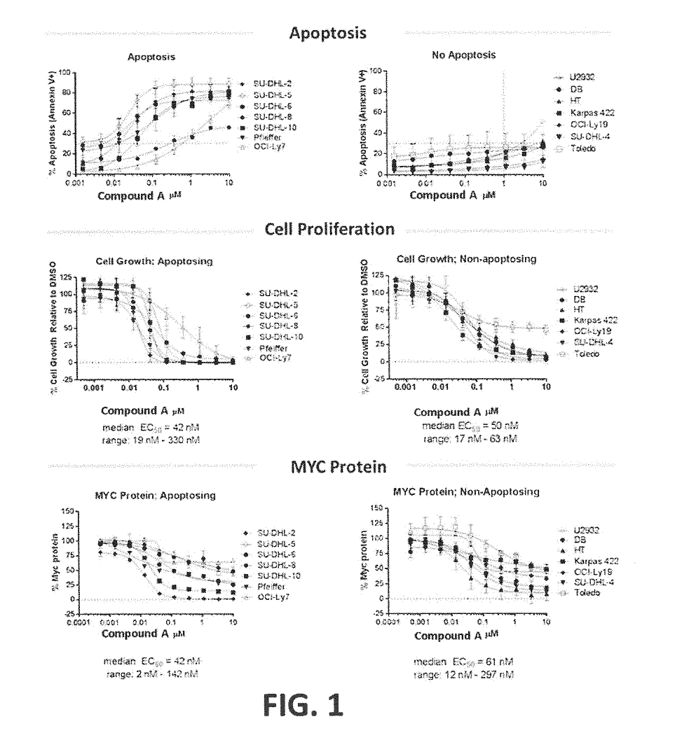

A subset of the lines that were shown to either undergo apoptosis or not were selected for protein expression profiling to delineate possible mechanisms for the disparity in the apoptotic response. The growth inhibition and apoptosis response for these lines is depicted in FIG. 1. In this subset of lines, seven underwent apoptosis in response to Compound A, and 8 did not (% apoptosis <30% by 1 μM). All cell lines responded to growth inhibition with the BET inhibitor (mean EC₅₀ 40 nM, range 12-72 nM). Additionally, Compound A treatment reduced Myc protein expression dose-dependently in all cell lines with EC₅₀s≤155 nM except one with an EC₅₀ for Myc reduction of 284 nM.

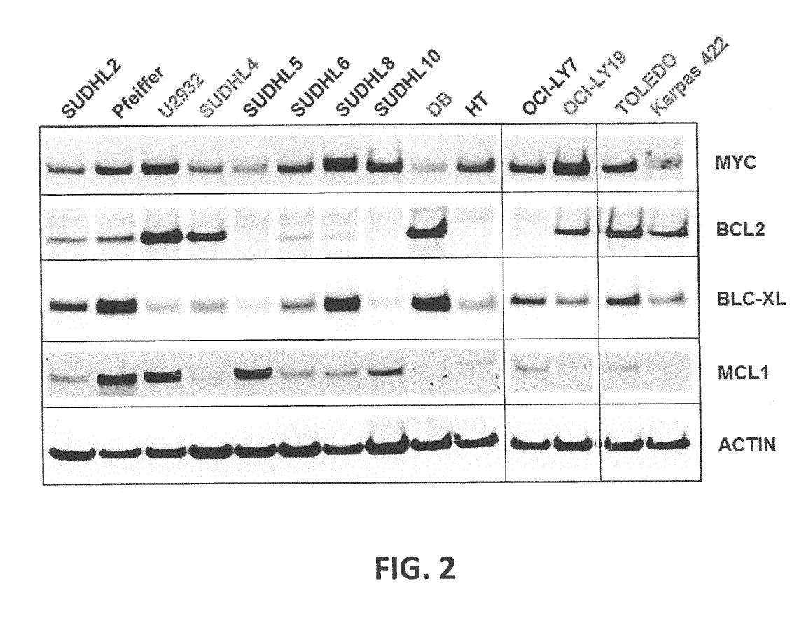

In order to evaluate whether the basal expression of Myc or the apoptotic machinery influenced the ability of BETi to induce apoptosis, the subset of lines in FIG. 1 were profiled for basal expression of Myc, Bcl-2, Mcl-1, Bim_{EL}, and Bcl-XL. Protein expression was quantified by Western Blot analysis of untreated lysates from 14 DLBCL and one FL cell line. The bands were quantified and normalized to actin expression and are listed in FIG. 2.

DLBCL cell lines were broadly sensitive to cell growth inhibition by Compound A, with EC₅₀s ranging from 17 to 330 nM (FIG. 1). Growth inhibition correlated with the reduction in Myc protein levels upon treatment with Compound A. Treatment with Compound A induced apoptosis in a subset of DLBCL cell lines (58%), which were enriched for those with low levels of Bcl-2 protein (FIG. 2).

### Example 3. High Basal Bcl-2 Protein Expression and Sensitivity to Venetoclax Correlate with Resistance to Apoptosis Induced by Single Agent Compound A

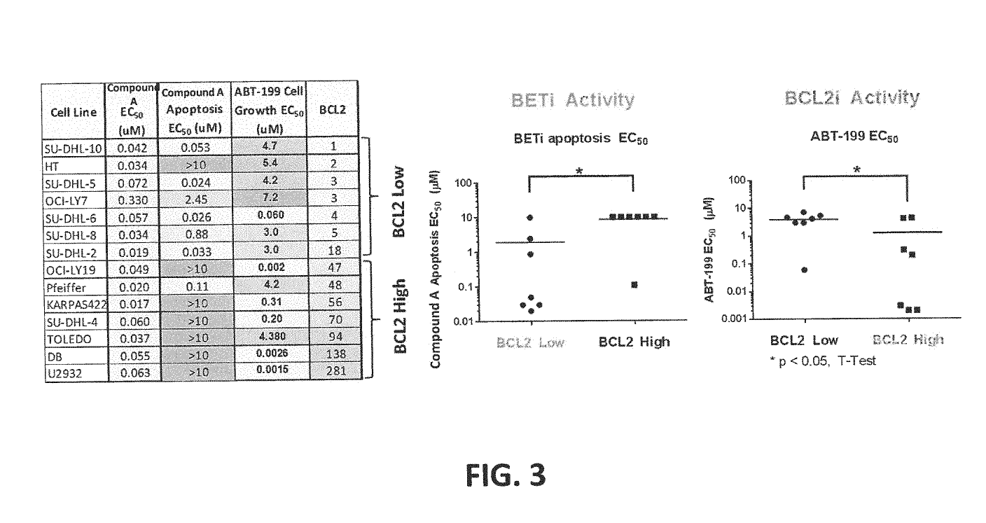

In order to understand the relationship between the basal expression of Myc and/or the apoptotic machinery and the ability of BET inhibitor (BETi) to induce apoptosis in the DLBCL and FL cell lines, values quantifying both the percent of apoptotic cells as well as the EC₅₀s for apoptosis in response to BETi were compared to the quantified protein expression. The “% apoptosis at Compound A EC₅₀” represents the percent of Annexin V positive cells at the EC₅₀ value for apoptosis minus the percent of Annexin V positive cells in untreated cells (i.e., baseline apoptosis) (FIG. 3).

Correlation of protein expression with apoptosis in response to treatment with Compound A was accomplished by separating the cell lines into those with high or low Bcl-2 expression (cutoff=20) and performing a T-Test comparing the activity of BETi between the two groups. Bcl-2 protein expression negatively correlated (p<0.05, T-Test) with the ability of Compound A to induce apoptosis, suggesting that high basal Bcl-2 protein expression prevented Compound A from inducing apoptosis in these lines. Expression of the other proteins was not significantly correlated with the ability of Compound A single agent to induce apoptosis.

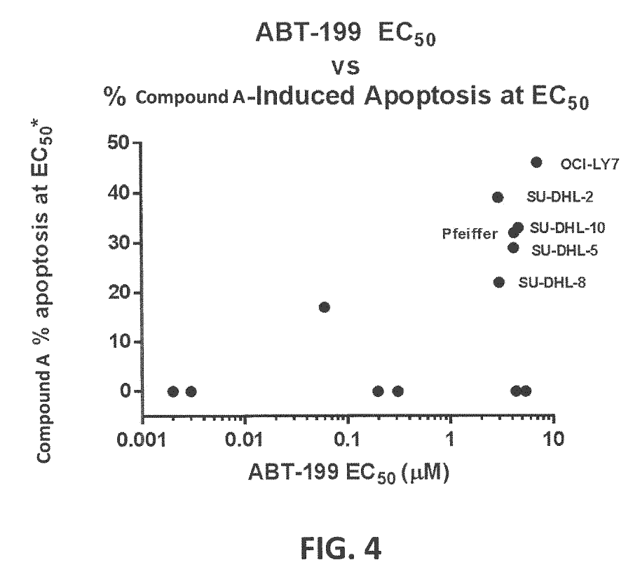

Because of the anti-correlation between high Bcl-2 expression and Compound A induced apoptosis, the Bcl-2 inhibitor, venetoclax, was evaluated for the ability to inhibit the cell growth across the same panel of one FL and 14 DLBCL cell lines and EC₅₀ values were determined. Bcl-2 protein expression positively correlated with sensitivity to venetoclax (p<0.05, T-Test). The significant difference between the EC₅₀s of the two inhibitors (of viability for venetoclax or apoptosis for Compound A) in DLBCL cell lines with either high or low basal Bcl-2 protein expression was depicted in FIG. 3. Apoptosis induction in response to Compound A and growth inhibition in response to venetoclax were compared in high and low Bcl-2 protein expression groups. DLBCL cell lines varied in sensitivity to venetoclax, with EC₅₀s ranging from 1.5 to 7200 nM (FIG. 3). Cell lines that were sensitive to venetoclax in general had high levels of Bcl-2 protein. 6 of the 8 lines that are refractory to Bcl-2 inhibition undergo apoptosis in response to Compound A (FIG. 4). DLBCL cell lines had a reciprocal sensitivity to the single-agent BET inhibitor Compound A and the Bcl-2 inhibitor, venetoclax.

In summary, those cell lines that responded to venetoclax (enriched for those expressing high levels of Bcl-2 protein) were less likely to respond apoptotically to Compound A. Conversely, those cell lines refractory to venetoclax (enriched for those expressing low levels of Bcl-2) were likely to be induced to undergo apoptosis with single agent Compound A (FIGS. 3-4). A combination of the two inhibitors would therefor increase the breadth of responding cell lines. This suggests that a broader patient population would respond to treatment with the combination than with either single agent alone.

### Example 4. Reduction of Myc in Response to Compound B

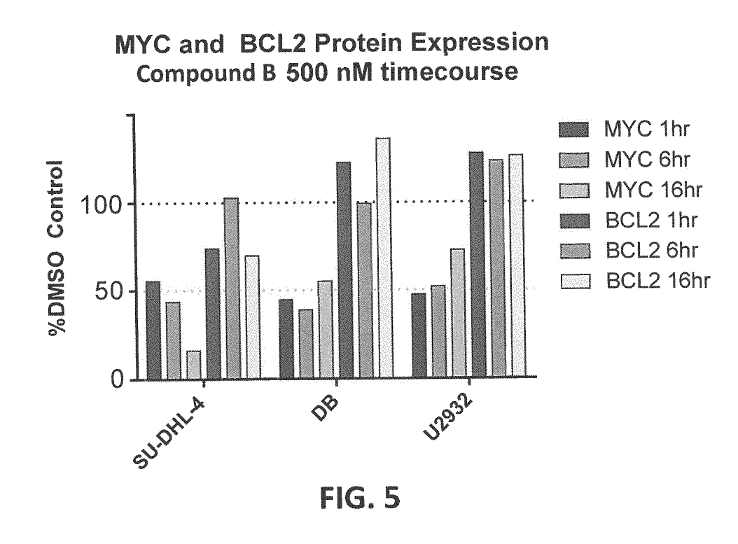

Myc and Bcl-2 protein levels in response to Compound B were studied in Human DLBCL cell lines that had high Bcl-2 levels. Compound B reduced the levels of Myc but not the levels of Bcl-2 in DLBCL cell lines expressing high levels of Bcl-2 (FIG. 5).

### Example 5. Combination of Venetoclax and Compound A

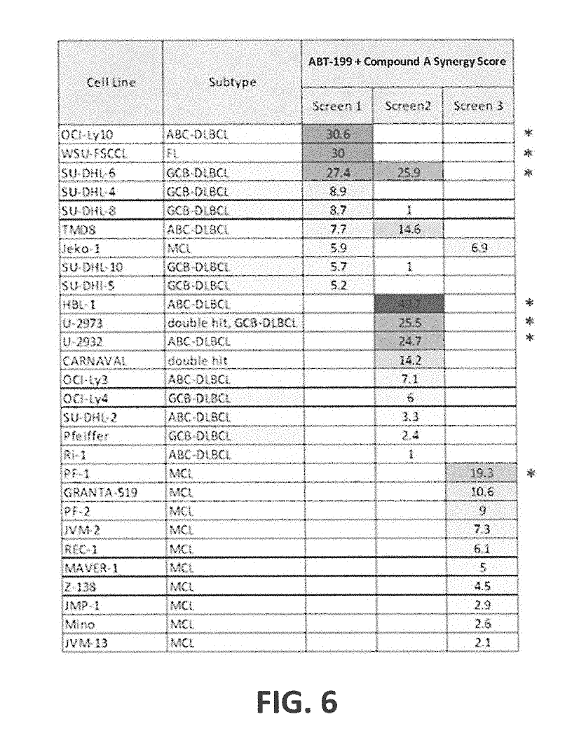

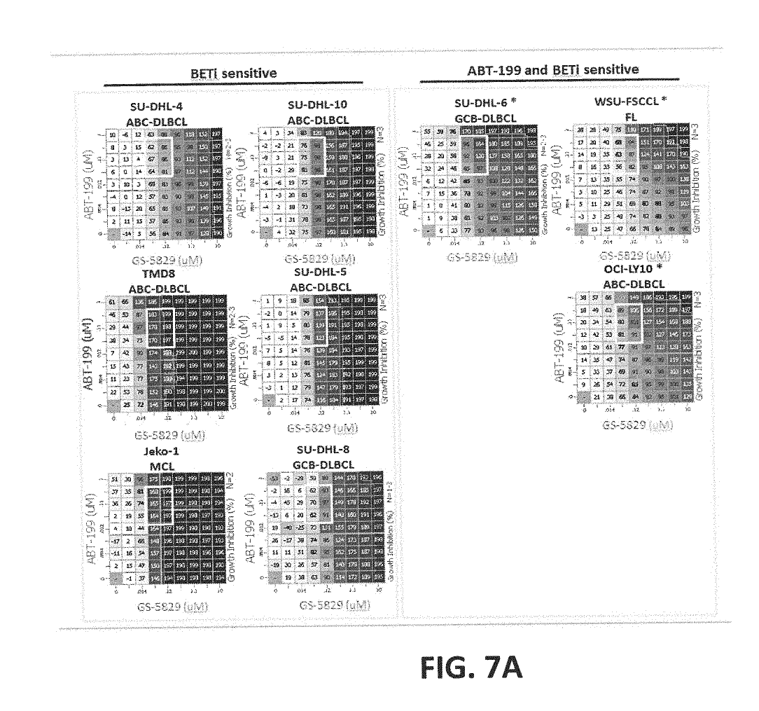

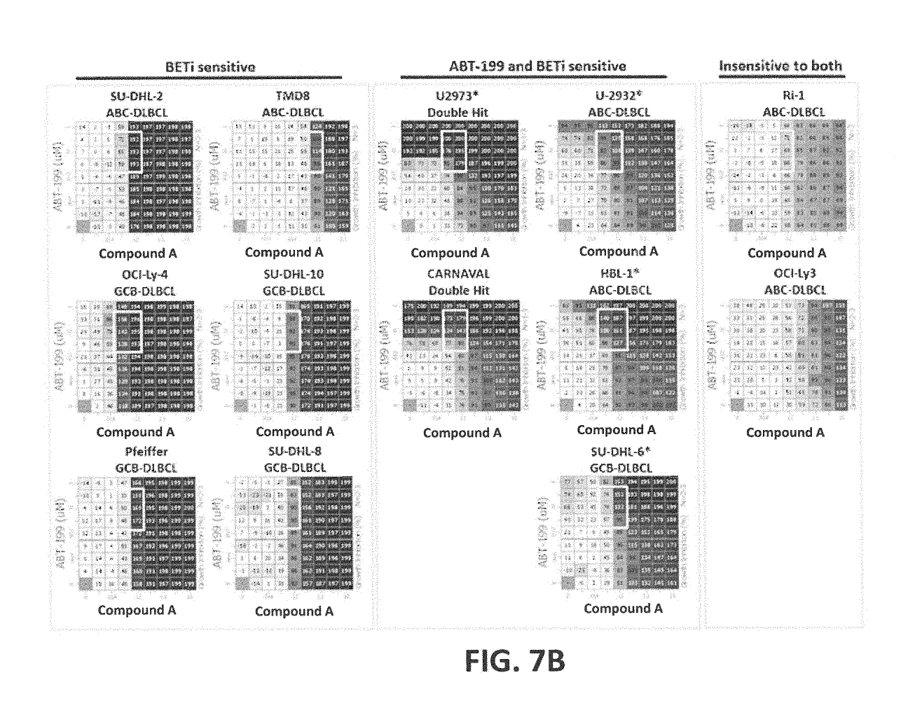

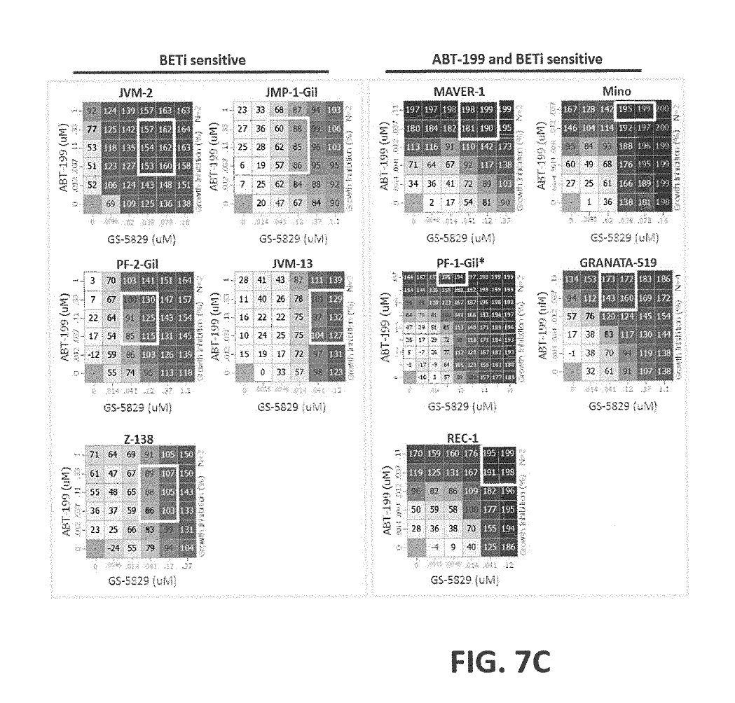

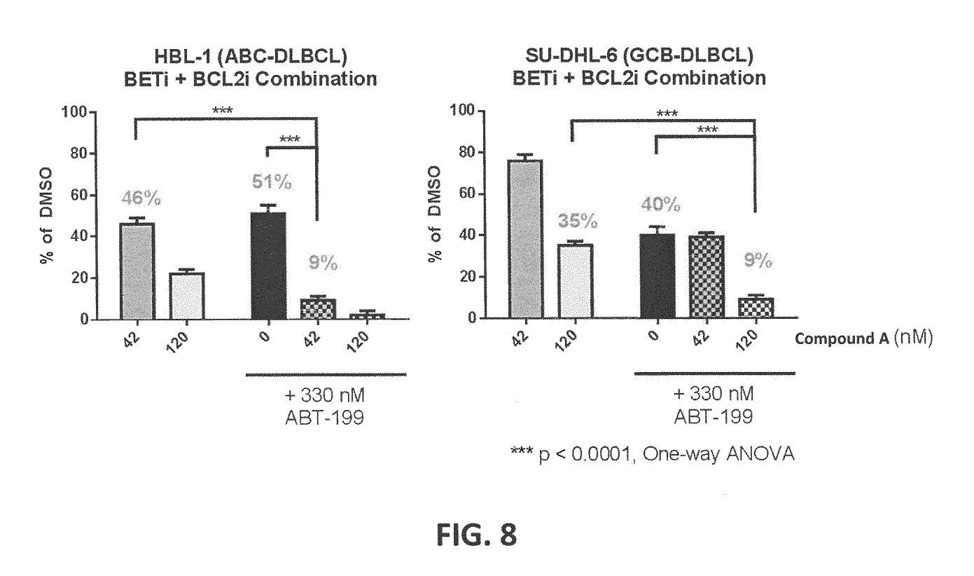

The data in the abovementioned experiments suggested that the combination of a Bcl-2 inhibitor and a BET inhibitor would broaden the number of patients responding to treatment. Therefor the following experiments tested the depth of the response in lymphoma cell lines to the two drugs administered simultaneously in vitro. Three identical synergy screens were performed on three different panels of lymphoma cell lines (FIGS. 7A-C). In all screens, cell lines were treated for 72 hrs and viability was measured by ATPLite which quantifies ATP as an indicator of viable cells. First, a panel of 1 FL, 1 MCL, and 7 DLBCL cell lines were screened. Second, a panel of 13 DLBCL cell lines were screened, 2 of which were also tested in the first screen. Third, a panel of 11 MCL cell lines were screened, one of which had been tested in the first screen. Synergy was calculated using a proprietary synergy calculation of the excess over Leowe additivity. The synergy scores generated for each cell line in each screen listed in FIG. 6. Cell lines with synergy scores >15 are marked with an asterix. Bar graphs depicting cell growth inhibition across the clinically relevant dose range for HBL-1 (synergy score 49.7) and SU-DHL-6 (synergy score 25.9) were drawn from data from screen 2 and are shown in FIG. 8.

Heatmaps depicting the extent of cell growth inhibition for all pairwise combinations were depicted in FIGS. 7A-C. The extent of cell growth inhibition in each square is denoted by color, as well as by a number, and represents the average of triplicate experiments. DMSO-treated and Day 0 measurements were acquired and used to establish no cell growth inhibition (a value of 0, yellow) and complete cell growth inhibition (a value of 100, red), respectively. Values >100 denote cell death resulting in fewer cells than were present at the beginning of treatment and ranged from 100 (the same as the Day 0 value, red) to 200 (complete absence of viable cells, black). Clinically relevant doses are depicted by white boxes encompassing the C_{trough} to Cₘₐₓ of each molecule. Derivations of the CCM protein adjusted Cₘₐₓ and Cₘᵢₙ concentrations are described in the methods.

A set of data was selected from the synergy screening to represent clinically relevant concentration ranges of venetoclax and Compound A. The average concentration of venetoclax (Cₐᵥ) was estimated at the 400 mg dose adjusted for protein binding using empirically determined free fraction in human plasma/cell culture media. Cₘᵢₙ and C_{trough} of Compound A were similarly adjusted.

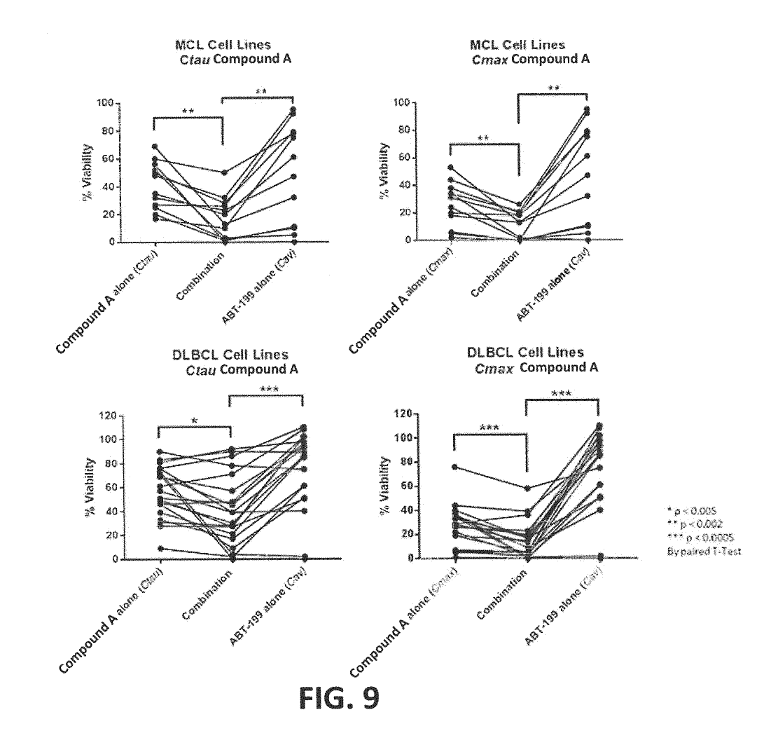

In order to understand whether the DLBCL or MCL cell line panels as a whole responded more favorably to the combination of clinically relevant concentrations of BETi with Bcl-2i than to either single agent alone, the percent cell growth inhibition for the estimated average clinical concentration (Cₐᵥ) of venetoclax single agent and both the CCM adjusted Cₜₐᵤ and Cₘₐₓ Compound A single agent were compared to the venetoclax Ca, in combination with either Cₜₐᵤ or Cₘₐₓ Compound A (FIG. 9). For both MCL and DLBLC cell line panels, at both Cₜₐᵤ and Cₘₐₓ concentrations of Compound A, the combination significantly increased the cell growth inhibition capacity over either single agent alone by paired T-Test (p<0.005).

The combination of Compound A and venetoclax at clinically relevant concentrations resulted in broader activity and greater suppression of cell growth than either inhibitor alone in DLBCL cell lines (FIGS. 8 and 9) and MCL cell lines (FIG. 9). The combination of Compound A and venetoclax resulted in superior growth inhibition in DLBCL and MCL cell lines, suggesting that such combination could lead to broader, superior responses in patients.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

What is claimed is:

1. A composition comprising a Bcl-2 inhibitor and a bromodomain and extra-terminal (BET) inhibitor.

2. The composition of claim 1, wherein the Bcl-2 inhibitor is selected from a group consisting of: ABT-199 (venetoclax), ABT-737, ABT-263 (navitoclax), AT-101 (Gossypol), apogossypol, TW-37, G3139 (Genasense or oblimersen), obatoclax, sabutoclax, HA14-1, antimycin A, S44563, and combinations thereof.

3. The composition of claim 1 or 2, wherein the Bcl-2 inhibitor is venetoclax.

4. The composition of claim 1 or 2, wherein the Bcl-2 inhibitor is ABT-737.

5. The composition of claim 1 or 2, wherein the Bcl-2 inhibitor is navitoclax.

6. The composition of any one of claims 1-5, wherein the BET inhibitor is a compound of Formula (Ib): 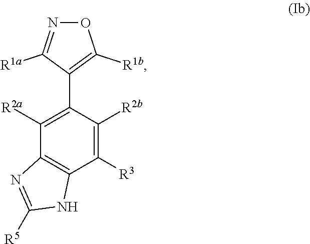
wherein
R^{1a} and R^{1b} are each independently C₁₋₆ alkyl optionally substituted with from 1 to 5 R² groups;
R^{2a} and R^{2b} are each independently H or halo;
R³ is
—C(O)OR^{a}, —NHC(O)OR^{a}, —NHS(O)₂R^{a}, or —S(O)₂NR^{a}R^{b}; or
selected from the group consisting of C₁₋₁₀ alkyl, C₁₋₁₀ alkoxy, amino, C₅₋₁₀ aryl, C₆₋₂₀ arylalkyl, C₁₋₁₀ heteroalkyl, C₅₋₁₀ heteroaryl, and C₆₋₂₀ heteroarylalkyl, each of which is optionally substituted with from 1 to 5 R²⁰ groups;
R is
—C(O)OR^{a}, —NHC(O)OR^{a}, —NHS(O)₂R^{a}, or —S(O)₂NR^{a}R^{b}; or
selected from the group consisting of H, C₁₋₁₀ alkyl, C₁₋₁₀ haloalkyl, C₁₋₁₀ alkoxy, amino, C₅₋₁₀ aryl, C₆₋₂₀ arylalkyl, C₁₋₁₀ heteroalkyl, C₁₋₁₀ heteroaryl, and C₆₋₂₀ heteroarylalkyl, each of which is optionally substituted with from 1 to 5 R²⁰ groups;
each R^{a} and R^{b} is independently selected from the group consisting of H, C₁₋₁₀ alkyl, C₅₋₁₀ aryl, C₆₋₂₀ arylalkyl, C₁₋₁₀ heteroalkyl, C₁₋₁₀ heteroaryl, and C₆₋₂₀ heteroarylalkyl, each of which is optionally substituted with from 1 to 5 R²⁰ groups; and
each R²⁰ is independently selected from the group consisting of acyl, C₁₋₁₀ alkyl, C₁₋₁₀ alkoxy, amino, amido, amidino, C₅₋₁₀ aryl, C₆₋₂₀ arylalkyl, azido, carbamoyl, carboxyl, carboxyl ester, cyano, guanidino, halo, C₁₋₁₀ haloalkyl, C₁₋₁₀ heteroalkyl, C₅₋₁₀ heteroaryl, C₆₋₂₀ heteroarylalkyl, hydroxy, hydrazino, hydroxyl, imino, oxo, nitro, sulfinyl, sulfonic acid, sulfonyl, thiocyanate, thiol, and thione;
wherein the C₁₋₁₀ alkyl, C₅₋₁₀ aryl, C₆₋₂₀ arylalkyl, C₁₋₁₀ heteroalkyl, C₁₋₁₀ heteroaryl, and C₆₋₂₀ heteroarylalkyl groups are optionally substituted with from 1 to 3 substituents independently selected from C₁₋₆ alkyl, C₅₋₁₀ aryl, halo, C₁₋₆ haloalkyl, cyano, hydroxyl, and C₁₋₆ alkoxy;
or a pharmaceutically acceptable salt, complex, solvate, prodrug, stereoisomer, mixture of stereoisomers or hydrate thereof.

7. The composition of claim 6, wherein R^{1a} and R^{1b} are each independently C₁₋₆ alkyl.

8. The composition of claim 6 or 7, wherein R³ is C₁₋₁₀ alkyl, C₁₋₁₀ alkoxy, or C₁₋₁₀ heteroalkyl, each of which may be optionally substituted with from 1 to 5 R²⁰ groups.

9. The composition of claim 6 or 7, wherein R³ is an, C₅₋₁₀ aryl, C₆₋₂₀ arylalkyl, C₅₋₁₀ heteroaryl, or C₆₋₂₀ heteroarylalkyl, each of which may be optionally substituted with from 1 to 5 R²⁰ groups.

10. The composition of any one of claims 6-9, wherein R⁵ is C₁₋₁₀ alkyl.

11. The composition of any one of claims 6-9, wherein R⁵ is C₁₋₁₀ haloalkyl.

12. The composition of any one of claims 6-9, wherein R⁵ is C₁₋₁₀ cycloalkyl.

13. The composition of claim 6, wherein the compound is of Formula (Ic): 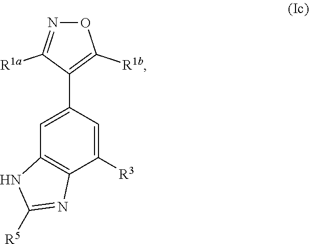
or a pharmaceutically acceptable salt, complex, solvate, prodrug, stereoisomer, mixture of stereoisomers or hydrate thereof.

14. The composition of claim 6, wherein the compound is of Formula (Id): 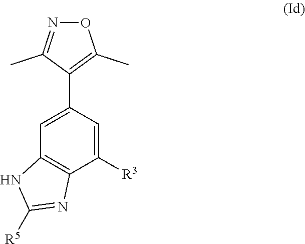
or a pharmaceutically acceptable salt, complex, solvate, prodrug, stereoisomer, mixture of stereoisomers or hydrate thereof.

15. The composition of claim 14, wherein R³ is C₁₋₁₀ alkyl, C₁₋₁₀ alkoxy, or C₁₋₁₀ heteroalkyl, each of which may be optionally substituted with from 1 to 5 R² groups.

16. The composition of claim 14, wherein R³ is an, C₅₋₁₀ aryl, C₆₋₂₀ arylalkyl, C₅₋₁₀ heteroaryl, or C₆₋₂₀ heteroarylalkyl, each of which may be optionally substituted with from 1 to 5 R²⁰ groups.

17. The composition of any one of claims 14-16, wherein R⁵ is C₁₋₁₀ alkyl.

18. The composition of any one of claims 14-16, wherein R⁵ is C₁₋₁₀ haloalkyl.

19. The composition of any one of claims 14-16, wherein R⁵ is C₁₋₁₀ cycloalkyl.

20. The composition of any one of claims 1-19, wherein the BET inhibitor is a compound of Table 1.

21. A composition comprising (i) a Bcl-2 inhibitor and (ii) Compound A, having the following formula: 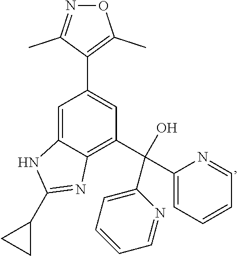 [SMILES: Cc1noc(C)c1-c1cc(C(O)(c2ccccn2)c2ccccn2)c2nc(C3CC3)[nH]c2c1]
or a pharmaceutically acceptable salt, complex, solvate, prodrug, stereoisomer, mixture of stereoisomers or hydrate thereof.

22. A composition comprising (i) venetoclax and (ii) Compound A, having the following formula: 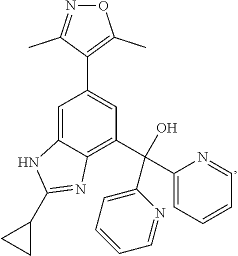 [SMILES: Cc1noc(C)c1-c1cc(C(O)(c2ccccn2)c2ccccn2)c2nc(C3CC3)[nH]c2c1]
or a pharmaceutically acceptable salt, complex, solvate, prodrug, stereoisomer, mixture of stereoisomers or hydrate thereof.

23. A composition comprising (i) venetoclax, and (ii) a bromodomain and extra-terminal (BET) inhibitor.

24. A method for treating cancer in a human patient in need thereof, comprising administering to the patient a therapeutically effective amount of the composition of any one of claims 1-23.

25. A method for treating cancer in a human patient in need thereof, comprising administering to the patient (i) a Bcl-2 inhibitor, and (ii) a bromodomain and extra-terminal (BET) inhibitor.

26. The method of claim 25, wherein the BET inhibitor is administered prior to, after, or concurrently with the Bcl-2 inhibitor.

27. A method for treating cancer in a human patient in need thereof, comprising administering to the patient (i) a Bcl-2 inhibitor, and (ii) Compound A, having the following formula: 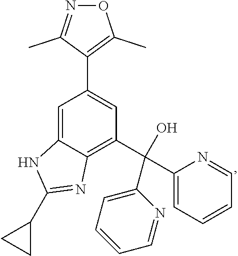 [SMILES: Cc1noc(C)c1-c1cc(C(O)(c2ccccn2)c2ccccn2)c2nc(C3CC3)[nH]c2c1]
or a pharmaceutically acceptable salt, complex, solvate, prodrug, stereoisomer, mixture of stereoisomers or hydrate thereof.

28. A method for treating cancer in a human patient in need thereof, comprising administering to the patient (i) venetoclax, and (ii) a bromodomain and extra-terminal (BET) inhibitor.

29. A method for treating cancer in a human patient in need thereof, comprising administering to the patient (i) venetoclax, and (ii) Compound A, having the following formula: 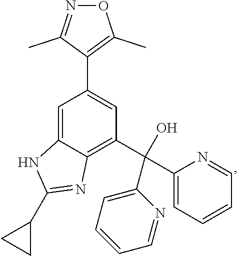 [SMILES: Cc1noc(C)c1-c1cc(C(O)(c2ccccn2)c2ccccn2)c2nc(C3CC3)[nH]c2c1]
or a pharmaceutically acceptable salt, complex, solvate, prodrug, stereoisomer, mixture of stereoisomers or hydrate thereof.

30. The method of any one of claims 24-29, wherein the cancer is a hematologic malignancy.

31. The method of any one of claims 24-29, wherein the cancer is a lymphoma.

32. The method of any one of claims 24-29, wherein the cancer is small lymphocytic lymphoma, non-Hodgkin's lymphoma, indolent non-Hodgkin's lymphoma, refractory iNHL, mantle cell lymphoma, follicular lymphoma, lymphoplasmacytic lymphoma, marginal zone lymphoma, immunoblastic large cell lymphoma, lymphoblastic lymphoma, Splenic marginal zone B-cell lymphoma (+/−villous lymphocytes), Nodal marginal zone lymphoma (+/−monocytoid B-cells), extranodal marginal zone B-cell lymphoma of mucosa-associated lymphoid tissue type, cutaneous T-cell lymphoma, extranodal T-cell lymphoma, anaplastic large cell lymphoma, angioimmunoblastic T-cell lymphoma, mycosis fungoides, B-cell lymphoma, diffuse large B-cell lymphoma, Mediastinal large B-cell lymphoma, Intravascular large B-cell lymphoma, Primary effusion lymphoma, small non-cleaved cell lymphoma, Burkitt's lymphoma, multiple myeloma, plasmacytoma, acute lymphocytic leukemia, T-cell acute lymphoblastic leukemia, B-cell acute lymphoblastic leukemia, B-cell prolymphocytic leukemia, acute myeloid leukemia, chronic lymphocytic leukemia, juvenile myelomonocytic leukemia, minimal residual disease, hairy cell leukemia, primary myelofibrosis, secondary myelofibrosis, chronic myeloid leukemia, myelodysplastic syndrome, myeloproliferative disease, or Waldestrom's macroglobulinemia.

33. The method of any one of claims 24-29, wherein the cancer is diffuse large B-cell lymphoma (DLBCL).

34. The method of any one of claims 24-29, wherein the cancer is follicular lymphoma (FL).

35. The method of any one of claims 24-34, wherein the cancer has overexpression of Myc.

36. The method of any one of claims 24-35, wherein the cancer has overexpression of Bcl-2.

37. The method of any one of claims 24-36, wherein the cancer has a translocation of Myc.

38. The method of any one of claims 24-37, wherein the cancer has a translocation of Bcl-2.

39. The method of any one of claims 24-29, wherein the patient has DLBCL or FL with overexpression or translation of Myc, Bcl-2 or the combination thereof.

40. A kit comprising (i) a pharmaceutical composition comprising a Bcl-2 inhibitor, (ii) a pharmaceutical composition comprising a BET inhibitor, and (iii) instructions for use of the Bcl-2 inhibitor and the BET inhibitor in treating cancer.
